(19) United States
(12) Patent Application Publication
(10) Pub. No.: US 2026/0192001 A1
(43) Pub. Date: Jul. 9, 2026

(54) FLUORINE METABOLIC IMAGING (FMI) WITH 3-FLUORO-3-DEOXY-SUGARS

(71) Applicants: YEDA Research and Development Co. Ltd., Rehovot (IL); I NEXT TECH ICHILOV LTD., Tel Aviv-Yafo (IL)

(72) Inventors: Amnon BAR-SHIR, Kibbutz Ramat HaKovesh (IL); Balamurugan SUBRAMANI, Rehovot (IL); Dan COHEN, Rehovot (IL)

(21) Appl. No.: 19/418,319

(22) Filed: Dec. 12, 2025

Related U.S. Application Data
(60) Provisional application No. 63/742,666, filed on Jan. 7, 2025.
(60) Provisional application No. 63/865,673, filed on Aug. 18, 2025.

(51) Int. Cl. A61K 51/04; A61P 35/00

(52) U.S. Cl. CPC A61K 51/0491; A61P 35/00; A61K 2123/00

(57) ABSTRACT

A metabolic imaging method based on ¹⁹F-MRI, comprising the steps of:
administering one or more 3-fluoro-3-deoxy-sugar (s) to a subject; applying ¹⁹F-magnetic resonance imaging (¹⁹F-MRI) to identify generation, localization and/or distribution of one or more fluorinated compounds in internal organs of the subject; and
assessing and/or diagnosing and/or monitoring a disease state associated with a pattern of abnormal activity of oxidizing and reducing metabolic enzymes.

### BACKGROUND OF THE INVENTION

There are different ways of introducing fluorine atoms to sugars, to form fluorinated sugars. For example, deoxyfluorination is the replacement of the hydroxyl group of the natural sugar by a fluorine atom. The so-formed sugars are named deoxyfluorinated sugars.

Deoxyfluorinated sugars can be used as probes in different fields, including sugar metabolic imaging. Instead of the natural sugar, a fluorinated analogue can be administered to the patient for use as a probe, to benefit from the detectability of the fluorine atom. For a metabolic imaging method to be useful in the diagnosis of a range of medical conditions, the fluorinated sugar should be taken up by cells, and metabolized via metabolic pathway(s) by one or more enzymes to produce at least one imageable metabolite that remains for sufficient time in the cells at appropriate level, indicating spatial localization and distribution of enzyme activities that can be linked to specific conditions or disease states.

In principle, ¹⁸F and ¹⁹F-labeled deoxyfluorinated sugars can be used as probes. ¹⁸F is the radioactive isotope, detectable by positron emission tomography (PET). ¹⁹F is the naturally occurring isotope, which can be detected by magnetic resonance imaging (MRI) owing to the magnetic properties of its nucleus. A deoxyfluorinated sugar used in metabolic imaging is 2-deoxy-2-[¹⁸F]-fluoro-D-glucose (abbreviated 2-[¹⁸F]FDG), which is the most common radioactive tracer in PET, whereas sugar metabolic ¹⁹F-MRI imaging method has not yet reached the clinic. Hereinafter, unless specifically indicated otherwise, the word fluorine, the prefix fluoro- and the like, indicate the natural isotope ¹⁹F.

2-deoxy-2-fluoro-sugars such as 2-[¹⁸F]FDG are phosphorylated upon entering the cell by the enzyme hexokinase to form the corresponding 6-phosphate metabolite akin to natural sugars. In contrast, sugar analogs that are deoxyfluorinated at the C-3 position interact differently with endogenous enzymes. It was shown that 3-fluoro-3-deoxy-sugars are metabolized via the polyol pathway, namely, they are reduced by aldose reductase to the corresponding polyol. For example, it was reported back in 1988 that 3-fluoro-3-deoxy-D-glucose (abbreviated 3FDG):

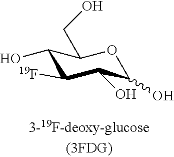

and 2-FDG were infused separately into rats. With the aid of ¹⁹F NMR spectroscopy it was shown that the main metabolite of 2-FDG was 2-FDG-6-phosphate, whereas 3-FDG was chiefly metabolized by aldose reductase to the corresponding reduced sugar form, i.e., the polyol 3-deoxy-3-fluoro-sorbitol (3FDS) (Nakada et al., “Fluorine-19 NMR Imaging of Glucose Metabolism”, Magnetic Resonance in Medicine, 6, 1988, 307-313). Another example is 3-fluoro-3-deoxy-D-galactose (abbreviated 3FDGal). This fluorinated sugar, with the structure depicted below:

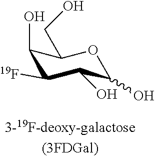

was shown to be useful in monitoring the activity of aldose reductase in dog lens by measuring with ¹⁹F-NMR spectroscopy the corresponding polyol metabolite 3-deoxy-3-fluoro-galactitol (3FGatol) (Secchi et al., “3-Fluoro-3-deoxy-D-galactose: A new probe for studies on sugar cataract”, Current Eye Research, 18(4), 1999, 277-82). The radioactive analogue, 3-[¹⁸F]-fluoro-3-deoxy-galactose was mentioned in U.S. Pat. No. 8,845,999.

### THE INVENTION

Experimental work conducted in support of this invention shows that not only 3-fluoro-3-deoxy-sugars are metabolized by reduction to the corresponding polyol via the aldose reductase pathway, but also by oxidation to the corresponding acids by the action of another enzyme (the enzyme that accounts for the formation of the acid an aldehyde dehydrogenase). The 3-fluoro-3-deoxy-sugars and their respective polyol and acid metabolites are collectively abbreviated herein as 3FD-sugars, 3FD-polyols and 3FD-acids, respectively.

For example, the polyol and acid metabolites of the two specific sugars mentioned above, 3FDG and 3FDGal, are shown in FIG. 1. The metabolites of 3FDG are 3-¹⁹F-deoxy-D-sorbitol and 3-¹⁹F-deoxy-gluconic acid (abbreviated herein 3FDS and 3FDGA, respectively). The metabolites of 3FDGal are 3-¹⁹F-deoxy-D-galactiol and 3-¹⁹F-deoxy-galactonic acid (abbreviated herein 3FDGtol and 3FDGalA, respectively). FIG. 1 also shows the phosphorylated metabolites of the two natural sugars (glucose and galactose), attesting to the significance of incorporating the fluorine atom at position 3, resulting in shifting the metabolism of the fluorinated sugars to different pathways.

Further experimental results reported below indicate that 3FD-sugar and its pair of metabolites are distinguishable from each other under ¹⁹F-NMR spectroscopy, i.e., the ¹⁹F-NMR shifts of 3FD-sugar and its 3FD-polyol and 3FD-acid metabolites differ one from another, making it possible to map simultaneously their distribution in organs with multiplexed ¹⁹F-MRI as demonstrated below.

For example, FIG. 2 shows the results of phantom experiments with tubes containing aqueous solutions of either 3FDG, 3FDGA or 3FDS, and likewise, a set of tubes containing aqueous solutions of 3FDGal, 3FDGalA or 3FDGtol. The results indicate different ¹⁹F-NMR chemical shifts, i.e., the spectral differentiation of the ¹⁹F-MR signals of 3FDG, 3FDGA and 3FDS and likewise, of 3FDGal, 3FDGalA or 3FDGtol from one another. Additionally, images appended in FIG. 2 show that it is possible, with the aid of appropriate imaging technique, i.e., an appropriate pulse sequence design, to spatially map the location of all ¹⁹F-compounds with a single ¹⁹F-MRI acquisition, visualizing ¹⁹F-labeled sugar and its pair of metabolites simultaneously. The pulse sequence which was used to acquire the ¹⁹F-MR images is a variant of the Fast Spin Echo (FSE) pulse sequence and is described in more detail below. Experimental work conducted in support of this invention also demonstrates the visualization of the two metabolites simultaneously after the transport of the 3-fluoro-3-deoxy-sugar into cells in vivo, thereby reporting the spatial distribution of sugar oxidizing and reducing enzymes in vivo and in real-time. That is, 3FD-sugars can be used for mapping metabolic pathways not detected nowadays with 2FDG.

Thus, one aspect of the invention is an imaging method for determining in vivo enzyme activity, comprising the steps of: administering one or more 3-fluoro-3-deoxy-sugar (s) to a subject; applying ¹⁹F-magnetic resonance imaging (¹⁹F-MRI) to identify generation, localization and/or distribution of fluorinated compounds in internal organs of the subject; and detecting the activity, or lack of activity, of oxidizing and reducing enzymes in said organs.

By the application of ¹⁹F-magnetic resonance imaging (¹⁹F-MRI), it is possible to identify the generation, location and/or distribution of the polyol and the acid metabolite of the 3-fluoro-3-deoxy-sugar (and/or downstream metabolites of the polyol and acid) in internal organ(s) of said subject.

Because two metabolites with different chemical shift spectra can be formed simultaneously upon administration of 3FD-sugar to a patient, namely, 3FD-sugar targets two different metabolic pathways provided by sugar oxidizing and reducing enzymes, then variation in reduction and oxidation enzymatic activity in different types of cells (e.g., cancerous cells) can create the unique signature of the combination of metabolites (i.e., neither 3FD-polyol nor 3FD-acid is formed, just one metabolite is formed, both 3FD-polyol and 3FD-acid are formed at different levels). Sometimes a high level of just one metabolite, either the 3FD-polyol or 3FD-acid, can be associated with disease state.

For example, in vitro incubation studies were conducted as reported below, in which seven types of cell lines were incubated with 3FDGal for a few hours, followed by ¹⁹F-NMR spectroscopy analysis of the extracellular medium (EC) and the intracellular content (IC). The spectra recorded based on the ¹⁹F-NMR signals of the metabolites 3FDGalA and 3FDGtol (distinct chemical shifts at −205 ppm and −211 ppm, respectively) showed cell-line specific ¹⁹F-NMR metabolic fingerprints, enabling categorization of the different types of cells based on 3FDGalA and 3FDGtol IC SNRs. The reduced (polyol) metabolite form was detected in varying concentrations in most cell lines studied, whereas the oxidized (acid) metabolite form was observed in two cell lines. Specifically, in A549 cells, both metabolites were detected, whereas in LNCaP cells, none was observed. Next, the 3FDGal metabolic variation was confirmed in vivo. Three athymic nude mice were subcutaneously injected with A549 and LNCap cells (both are cell lines of human origin) in the right and left posterior aspects of their chest, respectively, to induce proliferating subcutaneous tumors. Acquired in vivo 3D-¹⁹F metabolic imaging maps (with the aid of the imaging technique based on a modification of the RARE sequence) revealed both 3FDGalA and 3FDGtol generation by A549 tumors and no 3FDGal metabolism by LNCaP tumors, establishing the ability to distinguish between tumors at the molecular level with the aid of 3-fluoro-3-deoxy-sugars, such as 3FDGal.

This way, categorization of tumors becomes available based on their molecular composition, regardless of their dependence on glycolysis, simultaneously targeting two different metabolic pathways, and visualizing them in a multiplexed manner. As shown in the experimental work reported below, the human enzymes responsible for 3FDG and 3FDGal cellular metabolism, forming the acid and the polyol are aldehyde dehydrogenase (ALDH, e.g., ALDH1A1) and aldose reductase (AKR, e.g., AKR1B1), respectively.

Accordingly, the invention further provides an in vivo metabolic imaging method based on ¹⁹F-MRI, comprising the steps of: administering one or more 3-fluoro-3-deoxy-sugar (s) to a subject; applying ¹⁹F-magnetic resonance imaging (¹⁹F-MRI) to identify generation, localization and/or distribution of one or more fluorinated compounds in internal organs of the subject; and assessing and/or diagnosing and/or monitoring a disease state.

By the application of ¹⁹F-magnetic resonance imaging (¹⁹F-MRI), it is possible to identify the generation, location and/or distribution of the polyol and/or the acid metabolite of the 3-fluoro-3-deoxy-sugar (and/or downstream metabolites of the polyol and acid) in internal organ(s) of said subject, to assess a disease state, e.g., one that is associated with a pattern of abnormal activity of oxidizing and/or reducing enzymes, for example, diagnosing a tumor showing increased activity of both oxidizing and reducing enzymes.

The method of the invention further comprises a step of a 1H-MRI scan to create images of the internal organs of the examined subject, to identify the anatomical context of the ¹⁹F-labeled cells, and produce a merged image based on the 1H-MRI and ¹⁹F-MRI signals.

Another aspect of the invention relates to 3-fluoro-3-deoxy-sugar for use as an in vivo ¹⁹F-imaging agent in ¹⁹F-MRI assessment, diagnosis and/or monitoring of a disease that exhibits abnormal activity pattern of oxidizing and/or reducing enzymes, e.g., high level of one of such enzymes, or both, or increased activity of one, and reduced activity of the other.

That is, the diagnostic test provided by the invention is well suited for whole-body assessment and mapping of the expression of 3FD-sugars-reducing and -oxidizing enzymes, e.g., to detect lesions/pathologies/disease states that exhibit over-expression or under-expression of 3FD-sugars-reducing and -oxidizing enzymes, thereby enabling differentiation between lesions/pathologies/disease states at the molecular level (and not based on morphology).

Lesions/pathologies/disease states detectable, assessable and monitorable by the method of the invention include various cancers at any time point during the disease [at time of diagnosis (staging)], during or after different therapies (chemo, radio, immune, biological therapies), at time of disease recurrence, on remission.

Lesions/pathologies/disease states detectable, assessable and monitorable by the method of the invention include metabolic diseases, for example: metabolic dysfunction-associated fatty liver (MAFLD), diabetic complications including diabetic cardiomyopathy/neuropathy/nephropathy.

Lesions/pathologies/disease states that are detectable, assessable and monitorable by the method of the invention include also inflammatory/autoimmunity-related diseases, such as inflammatory bowel disease (IBD).

The method of the invention can help with the detection, assessment and monitoring of cancer types which are not well suited to [¹⁸F]2FDG PET (for example, cancers that do not follow the Warburg effect). Examples within this group include prostate cancer, renal cell carcinomas, ovarian cancers (and other gynecological cancers), some GI-cancers (particularly signet-ring/mucinous adenocarcinomas), pancreatic cancer, multiple myeloma and neuroendocrine cancers.

The method of the invention can help with the detection, assessment and monitoring of cancer types related to overexpression of 3FD-sugars-reducing and -oxidizing enzymes, such as renal cell carcinoma, pancreatic cancer and ovarian cancer.

For example, lung cancers (e.g., A549 tumors) are detectable, assessable and monitorable by the ¹⁹F-MRI diagnostic test provided by the invention because it shows increased sugar oxidation and reduction simultaneously. It is worth mentioning that the A549 cell line has been shown to have poor avidity to 2FDG. Renal cell carcinoma and brain tumors are also detectable, assessable and monitorable by the method of the invention.

Monitoring of a disease state, such as the progress of a metabolic disease, or the progress or remission of a cancer, can be aided by the present invention. For example, the efficacy of a treatment such as drug therapy can be assessed by the imaging method of the invention, e.g., an increase or decrease in enzyme activity can signify an improvement or worsening of the disease. Drug/radiation therapy and dosage regimen can be chosen with the aid of the present invention to guide clinical decisions.

3-fluoro-3-deoxy-sugars for use in the invention are preferably 3-fluoro-3-deoxy hexoses such as 3-fluoro-3-deoxy-D-glucose, 3-fluoro-3-deoxy-D-galactose, 3-fluoro-3-deoxy-D-mannose and corresponding methyl ether derivatives. Deoxyfluorinated hexoses for use in the invention are commercially available or can be synthesized by methods known in the art. For example, 3-fluoro-3-deoxy-D-galactose can be prepared from 1,2:5,6-di-O-isopropylidene-3-O-toluene-p-sulfonyl-α-D-gulofuranose by the chemical reaction described by Brimacombe et al. [“Fluorinated carbohydrates. Part VI. Studies of derivatives of 3-deoxy-3-fluoro-D-galactose”, Canadian Journal of Chemistry, 58, 1970, 3947-3952. Preparation of 3-fluoro-3-deoxy-D-glucose is described in Foster et al., Carbohydrate Research, 5 (1967), 292-301 and in Tewson et al., J. Org. Chem., Vol. 43, No. 6, 1978. The synthesis of 3-fluoro-3-deoxy-D-mannose is described in Rasmussen et al., Carbohydrate Research, 116, 1983 21-29. For example, 3-fluoro-3-deoxy pentoses such as 3-Fluoro-3-deoxy-D-xylose (3FDXyl) and 3-Fluoro-3-deoxy-D-ribose (3FDRib) are also useful; their synthesis is reported below. ¹⁹F-NMR spectra of 3FDXyl and its redox metabolites (the corresponding acid and polyol) indicate sufficiently distinct chemical shifts (data tabulated in Table 1 below).

Thus, 3-fluoro-3-deoxy-sugars for use in the invention can be selected from the group consisting of 3-Fluoro-3-deoxy-D-glucose (3FDG); 3-Fluoro-3-deoxy-D-galactose (3FDGal); 3-Fluoro-3-deoxy-D-mannose (3FDM); 3-Fluoro-3-deoxy-D-allose (3FDAllose); 3-Fluoro-3-deoxy-D-gulose (3FDGulose); 3-Fluoro-3-deoxy-D-talose (3FDTalose); 3-Fluoro-3-deoxy-D-idose (3FDIdose); 3-Fluoro-3-deoxy-D-altrose (3FDAltrose); 3-Fluoro-3-deoxy-D-xylose (3FDXyl); 3-Fluoro-3-deoxy-D-arabinose (3FDArab); 3-Fluoro-3-deoxy-D-ribose (3FDRib); 3-Fluoro-3-deoxy-D-lyxose (3FDLyxose); 3-Fluoro-3-deoxy-D-fructose (3FDFruct); 3-Fluoro-3-deoxy-D-sorbose (3FDSorbose); 3-Fluoro-3-deoxy-D-tagatose (3FDTagatose); 3-Fluoro-3-deoxy-D-psicose (3FDPsicose); 3-Fluoro-3-deoxy-D-threose (3FDThreose); 3-Fluoro-3-deoxy-D-erythrose (3FDErythrose); 3-Fluoro-3-deoxy-L-glucose (3FLG); 3-Fluoro-3-deoxy-L-galactose (3FLGal) 3-Fluoro-3-deoxy-L-altrose (3FLAltrose); 3-Fluoro-3-deoxy-L-talose (3FLTalose); 3-Fluoro-3-deoxy-L-idose (3FLIdose); 3-Fluoro-3-deoxy-L-xylose (3FLXyl); 3-Fluoro-3-deoxy-L-arabinose (3FLAra); 3-Fluoro-3-deoxy-L-ribose (3FLRib); 3-Fluoro-3-deoxy-L-threose (3FLThreose);
1-O-Methyl-3-fluoro-3-deoxy-D-glucose (1OMe-3FDG); 6-O-Methyl-3-fluoro-3-deoxy-D-glucose (6OMe-3FDG); 4-O-Methyl-3-fluoro-3-deoxy-D-galactose (4OMe-3FDGal); 6-O-Methyl-3-fluoro-3-deoxy-D-galactose (6OMe-3FDGal); 1-O-Methyl-3-fluoro-3-deoxy-D-mannose (1OMe-3FDM); 6-O-Methyl-3-fluoro-3-deoxy-D-mannose (6OMe-3FDM); 1-O-Methyl-3-fluoro-3-deoxy-D-xylose (1OMe-3FDXyl); 4-O-Methyl-3-fluoro-3-deoxy-D-xylose (4OMe-3FDXyl); 1-O-Methyl-3-fluoro-3-deoxy-D-ribose (1OMe-3FDRib); 1-O-Methyl-3-fluoro-3-deoxy-D-fructose (1OMe-3FDFruct);
3-Fluoro-3-deoxy-D-glucosamine (3FDGlcN); 3-Fluoro-3-deoxy-D-galactosamine (3FDGalN); 3-Fluoro-3-deoxy-N-acetylglucosamine (3FDGlcNAc); 3-Fluoro-3-deoxy-N-acetylgalactosamine (3FDGalNAc); 3-Fluoro-3-deoxy-D-mannosamine (3FDMannosamine); 3-Fluoro-3-deoxy-L-glucosamine (3FLGlcN); 3-Fluoro-3-deoxy-L-galactosamine (3FLGalN); 3-Fluoro-3-deoxy-D-fucosamine (3FDFucN); 3-Fluoro-3-deoxy-D-rhamnosamine (3FDRhamN); and 3-Fluoro-3-deoxy-N-acetylmannosamine (3FDMannNAc).

The experimental results reported below indicate that 3-fluoro-3-deoxy-D-galactose (3FDGal) is a highly useful ¹⁹F-imaging agent. The redox products of 3FDGal are not only spectrally distinct from one another (Δω~5 ppm) and directly related to e.g., ALDH1A1 (via 3FDGalA) and AKR1B1 (via 3FDGtol) activities, they are also both intracellularly stable (“trapped” and not further metabolized). 3FDGal was found to enable effective in vivo visualization of tissue-specific propensities for metabolic pathways initiated by sugar oxidation or reduction, more efficiently compared to other 3-fluoro-3-deoxy hexoses, such as 3-fluoro-3-deoxy-D-glucose (3FDG). The reason is the enhanced detectability of the acid metabolite of 3FDGal, 3-¹⁹F-deoxy-galactonic acid (3FDGalA), relative to the acid metabolite of 3DFG, 3FDGA. It appears that neither 3-¹⁹F-deoxy-galactonic acid nor 3-¹⁹F-deoxy-D-galactiol undergo further metabolism to produce downstream metabolites. In contrast, the reduced (polyol) form of 3FDG—3FDS—is further metabolized.

That is, data reported below shows that 3FDGal has the potential to trace two different redox biomarkers in, e.g., cancer, ALDH1A1 and AKR1B1, highlighting the superiority of 3FDGal over 3FDG in selectively reporting the spatial distribution of sugar oxidizing and reducing enzymes in vivo and in real-time. Additionally, the superiority of 3FDGal over 3FDG was verified in an enzyme-substrate kinetic assay (no cells or tissue involved), showing that AKR1B1 has indeed a lower Km (better affinity) to 3FDGal over 3FDG. Given the crucial role of sugar oxidation and reduction in a wide range of pathologies, from metabolic diseases through cancer reprogramming, 3FDGal can be used as an imaging agent to detect a wide range of conditions. The invention therefore specifically provides 3-fluoro-3-deoxy-D-galactose for use as a ¹⁹F-imaging agent in the diagnosis and monitoring of diseases (e.g., assessing the effect of drug/radiation therapy), e.g., metabolic diseases and also detection and monitoring cancer such as lung cancer, renal cancer and brain cancer (primary and secondary tumors) in vivo in a subject. Additionally, in vivo toxicity assessment showed no signs of toxicity on H&E staining method of kidneys, liver, heart, spleen, lungs, with normal comprehensive biochemical blood panels 4 and 24 h after 0.7 g/kg IV dose of 3FDGal.

3-Fluoro-3-deoxy-sugar, such as 3FDGal, will usually be administered to the patient parenterally, e.g., by intravenous injection, or through indwelling catheter or orally, e.g., as an aqueous solution. 3-fluoro-3-deoxy-sugar can also be formulated into a solid dosage form such as a tablet or capsule. A dose ranging from 0.1 to 1000 mg/kg body weight is considered appropriate for assessment, diagnosis and/or monitoring. Thus, another aspect of the invention is a parenterally or an orally administrable composition comprising 3-fluoro-3-deoxy-sugar and a carrier, for the uses indicated above, i.e., assessment, diagnosis and/or monitoring of diseases showing abnormal patterns of activity of oxidizing and reducing enzymes.

The invention provides an injectable formulation comprising an isotonic saline, sterile, optionally buffered solution of 3-fluoro-3-deoxy-sugar for the uses indicated above. For example, a solution for injection is prepared by dissolving in 10 to 100 ml of saline solution (optionally buffered; phosphate buffer, histidine buffer or citrate buffer) an effective amount of 3-fluoro-3-deoxy-sugar, i.e., an amount for delivering the desired dose of 0.1 to 1000 mg/kg body weight.

The invention provides aqueous solution for oral administration for the uses indicated above, comprising the 3-fluoro-3-deoxy-sugar dissolved in drinking water. For example, a solution is prepared by dissolving in 200 ml water an effective amount of 3-fluoro-3-deoxy-sugar, i.e., an amount for delivering the desired dose of 0.1 to 1000 mg/kg body weight.

The time interval between the administration of 3-fluoro-3-deoxy-sugar and the start of the MRI scan may be from 1 min to several hours, allowing sufficient uptake of the 3-fluoro-3-deoxy-sugar and its intracellular metabolism to take place. The MRI scan preferably starts at least one hour post injection, e.g., at least 1.5 hours, for example, about two to three hours post injection, with MRI scan time (i.e., acquisition time—the duration of the scan to collect data and form images) of less than 60 minutes, e.g., less than 45 minutes, e.g., from 15 to 30 minutes, ~20 minutes.

As pointed out above, besides the acquisition of the ¹⁹F spectral data and related images, the creation of a 1H-MRI image is desirable because it identifies the anatomical background of the ¹⁹F-labeled cells (for visualization of ¹⁹F-MRI FMI data overlaid on anatomical data). The ¹⁹F and ¹H spectral data can be acquired sequentially, in either order; eventually, the ¹⁹F image is merged with the ¹H anatomical image. But it is of course more efficient that spectral signals be collected for both ¹⁹F and ¹H in one imaging session, with the help of suitable double-resonant 1H/¹⁹F coils.

With appropriate modifications to the hardware and/or software, MRI scanners can be adapted to image ¹⁹F for use in the invention, that is, both data sets can be acquired with the same instrument. The adjustments required are known to those skilled in the art and are described in detail below in the experimental section in reference to a lab-scale setup based on a small-animal scanner, e.g., tuning and matching the ¹H/¹⁹F frequencies and magnetic shimming. The adaptation of ¹H/¹⁹F scanners is also described in two recently published papers: 1) van Heeswijk et al., “Cardiovascular Molecular Imaging With Fluorine-19 MRI: The Road to the Clinic”, Circ Cardiovasc Imaging, 16(9) 2023,e 014742, DOI: 10.1161/CIRCIMAGING.123.014742; and 2) Maxouri et al., “How to 19F MRI: applications, technique, and getting started”, BJR Open, 5(1), 2023, 20230019, DOI: 10.1259/bjro.20230019. Briefly, an MRI scanner needs to have a radiofrequency (RF) system that transmits and receives at the appropriate frequency to resonant ¹⁹F nucleus. For clinical MRI scanners, an RF amplifier or the addition of a suitable 1H/¹⁹F coil with appropriate geometry may be needed for the RF transmission and reception.

The subject is positioned in the scanner under the action of a magnetic field (e.g., in the 0.5 to 7 Tesla range) and pulsed radiofrequency (RF) is delivered. The choice of pulse sequence is important. Because multiple ¹⁹F-labeled metabolites are formed and need to be imaged, it is preferred to use a pulse sequence that can simultaneously acquire several frequencies to detect the individual ¹⁹F-labeled metabolites and concurrently record multiple metabolic images in a single scan. Suitable approaches are described, for example, by Flagel U et al. Multi-targeted ¹H/¹⁹F MRI unmasks specific danger patterns for emerging cardiovascular disorders. Nat Commun. 2021, 12:5847; van Heeswijk R B et al. Chemical shift encoding (CSE) for sensitive fluorine-19 MRI of perfluorocarbons with complex spectra. Magn Reson Med. 2018; 79:2724-2730; and Schoormans J et al. An iterative sparse deconvolution method for simultaneous multicolor ¹⁹F-MRI of multiple contrast agents. Magn Reson Med. 2020; 83:228-239.

Accordingly, another aspect of the invention is an imaging method based on multiplexed/multispectral ¹⁹F-MRI, which comprises simultaneously acquiring distinct ¹⁹F MR signals assigned to the individual ¹⁹F-compounds (polyol and acid metabolites of the 3-fluoro-3-deoxy-sugar, and/or downstream metabolites thereof) in a single imaging session and simultaneously visualizing their formation in internal organs.

The multiple ¹⁹F MR signals are preferably acquired by applying a pulse sequence that can excite multiple resonances in a single scan, e.g., with the aid of ¹⁹F-multi chemical shift selective imaging (mCSSI), as described by Flögel U (supra). Briefly, mCSSI is a RARE sequence variant that replaces traditional RARE's slice selection with frequency selective excitation, applying a narrow bandwidth excitation (by narrow is meant about 1 to 5 ppm, e.g., in the experimental work reported below, a RF bandwidth of 2000 Hz was applied for 9.4 Tesla magnetic field), followed by phase encoding of the third dimension, and acquires the echo train with selective refocusing pulses. At the end of each cycle, gradient spoiling destroys the remaining transverse magnetization. The entire procedure is repeated for each of the excited chemical shifts within a single Repetition Time (TR). This way, setting n ¹⁹F chemical shifts during one FMI step, n matrices are acquired (e.g., n=3 in the present invention, considering the administered 3-fluoro-3-deoxy-sugar transported into the cells and the pair of metabolites that can potentially be formed). Each matrix records per-voxel signal intensity that corresponds to the spatial distribution of a specific frequency (assigned to an individual fluorinated compound) on the 19F NMR spectrum. Images can be created from the data stored in the n matrices to simultaneously visualize the location of all fluorinated compounds.

The characteristic excitation frequencies associated with the exogenously applied sugar and the endogenously generated reduced (polyol) and oxidized (acid) metabolites can be recorded beforehand by non-localizing 19F-NMR. Characteristic sets of excitation frequencies for use by the method of the invention are tabulated in Table 1 below.

TABLE 1

| 3FD-sugar * | 3FD-polyol | 3FD-acid |
|---|---|---|
| 3FDGal at −198.3 ± 1 ppm | 3FDGtol at −209.9 ± 1 ppm | 3FDGalA at −205.1 ± 1 ppm |
| 3FDG at −194.2 ± 1 ppm | 3FDS at −212.4 ± 1 ppm | 3FDGA at −208.4 ± 1 ppm |
| 3FDXyl at −197 ± 1 ppm | 3FDXtol at −213 ± 1 ppm | 3FDXylA at −208 ± 1 ppm |

* the high-frequency beta anomer

Slight variations due to instrument calibration may occur; the differences in shifts are:
Δω (3FDGal-3FDGtol=11.6 ppm), Δω (3FDGal-3FDGa1A=6.8 ppm);
Δω (3FDG-3FDS=18.2 ppm), Δω (3FDG-3FDGA=14.2 ppm);
Δω (3FDXyl-3FDXtol=16 ppm), Δω (3FXyl-3FDXylA=11 ppm).

Thus, the invention further provides a method, wherein the 3-fluoro-3-deoxy sugar is 3-fluoro-3-deoxy-D-galactose, comprising recording and/or mapping signals at Δω=11.6±0.5 ppm and Δω=6.8±0.5 ppm, relative to the signal of the beta anomer of 3-fluoro-3-deoxy-D-galactose, respectively, corresponding to the reduced and oxidized metabolites of 3-fluoro-3-deoxy-D-galactose.

The parameters of the method may vary within the following ranges:
Repetition time (TR) from 100 to 8000 ms;
Echo time (TE)=from 1 to 200 ms;
Echo spacing=from 0.1 to 4 ms;
RARE factor (or Echo Trail Length, ETL)=from 1 to 32;
To record 2D images: [Averages=1-128, scan time=1-60 min], slices thicknesses: 1-100 mm; Field of view (FOV)=10 mm×10 mm to 50 cm×50 cm; matrix size=16×16 to 256×256; to result in an in-plane resolution of FOV/matrix size.
To record 3D FMI: [scan time=1-60 min], slices/thicknesses: 1-100 mm; Field of view (FOV)=10 mm×10 mm×10 mm to 50 cm×50×50 cm; matrix size=16×16×16 to 256×256×256; to result in a voxel size: of FOV/matrix size.

Accordingly, the multiplexed/multispectral ¹⁹F-MRI imaging method of the invention comprises acquiring distinct ¹⁹F MR signals assigned to ¹⁹F-compounds selected from:
the polyol metabolite of 3-fluoro-3-deoxy-sugar, and/or downstream metabolites of the polyol; and
the acid metabolite of the 3-fluoro-3-deoxy-sugar, and/or downstream metabolites of the acid; and
optionally the administered 3-fluoro-3-deoxy-sugar,
   in a single imaging session by applying a pulse sequence that can excite multiple resonances and recording per-voxel multiple signal intensities, each corresponding to a spatial distribution of a specific frequency assigned to an individual fluorinated compound.

Data visualization and color assignment are conducted by methods known in the art as demonstrated below.

The invention enables stratifying therapeutic strategy based on FMI findings. In vivo whole-body molecular imaging using FMI provides high sensitivity and specificity for detecting lesions, including lesions with normal morphology on CT or 1H-MRI. Because FMI of the invention enables sensitive identification of metastatic disease, it allows the physician to exclude certain patients from unnecessary surgical intervention.

The invention relates to a method of treatment, e.g., a personalized-medicine method for managing a disease state in a subject, wherein the disease is first determined to be aldose reductase (e.g., AKR1B1)-rich and/or aldehyde dehydrogenase (e.g. ALDH1A1)-rich (e.g., showing increased AKR1B1 and/or ALDH1A1 activity), and establishment of the relevant FMI fingerprint, for example based on positive immunohistochemical staining of an ex vivo sample (biopsy), comprising using in vivo FMI, assessing disease extent, and deciding on a therapy selected from surgery to remove a tumor, radiotherapy and/or systemic therapy.

For example, when FMI indicates localized disease without secondary lesions (i.e., absent of FMI signals spread over the body), then surgery and/or radiotherapy are the therapies of choice.

For example, when FMI indicates a locally-advanced disease (e.g., involvement of regional draining lymph-node stations), then the therapy of choice is surgery and/or radiotherapy.

For example, when FMI indicates an advanced, spread (metastatic) disease state, then the therapy of choice is a systemic treatment comprising administration of one or more active agents to the patient. For example, for AKR-positive disease, an aldose reductase inhibitor may be considered (e.g., Epalrestat, Ranirestat). For ALDH-positive disease, an aldehyde dehydrogenase inhibitor may be considered (e.g., disulfiram).

The FMI of the invention may be further used to monitor disease activity, e.g., routine monitoring of disease positivity during follow-up (e.g., annual surveillance after disease eradication), localization of disease sites when recurrence is clinically suspected (e.g., in the setting of non-localizing elevated blood biomarkers). The FMI of the invention specifically enables assessment of therapeutic efficacy, e.g., the benefit of administering AKR inhibitor and/or ALDH inhibitor.

Useful FMI fingerprints include high AKR/high ALDH, high AKR/low ALDH and low AKR/high ALDH. The fingerprint may be determined based on biopsy or literature. By way of example, the data shown below demonstrates that 7/8 different cells lines derived from renal cell carcinoma (RCC) are characterized by a redox biomarker profile of very high AKR fingerprint, with a variable expression of ALDH. Thus, even if just one of the above enzymes is overexpressed, the resulting metabolite thereof allows detection of cells suspicious as cancerous throughout the body. Additionally, it should be noted that based on the description above, in vitro and ex vivo diagnosis (also with the help of ¹⁹F-MRS) are provided by the invention.

The invention further provides an imaging method for determining in vivo enzyme activity, comprising the steps of:
administering 3-¹⁸F-3-deoxy-sugar radiotracer (e.g., 3-¹⁸F-3-deoxy-D-galactose or 3-¹⁸F-3-deoxy-D-xylose) to a subject; scanning the subject with positron emission tomography (PET) to identify generation, location and/or distribution of fluorinated compounds in internal organs and/or whole body of the subject; and
detecting the activity, or lack of activity, of 3FD-sugar-metabolizing enzymes in said organs and/or whole body.

The invention further provides an in vivo metabolic imaging method based on ¹⁸F-labeled radiotracer, comprising the steps of: administering 3-¹⁸F-3-deoxy-sugar radiotracer (e.g., 3-¹⁸F-3-deoxy-D-galactose or 3-¹⁸F-3-deoxy-D-xylose) to a subject; scanning the subject with positron emission tomography (PET) to identify generation, localization and/or distribution of fluorinated compounds in internal organs and/or whole body of the subject; and assessing and/or diagnosing and/or monitoring a disease state.

By the application of PET, it is possible to detect the (total) signal produced by one or more of the following fluorinated compounds (on PET, the probe and all metabolites emit the same indistinguishable signal):
polyol metabolite of the 3-¹⁸F-3-deoxy-sugar;
downstream metabolites of the polyol;
acid metabolite of the 3-¹⁸F-3-deoxy-sugar;
downstream metabolites of the acid; and
the administered probe, i.e., the 3-¹⁸F-3-deoxy-sugar;
   to identify the generation and/or localization and/or distribution of the polyol and/or the acid metabolite of the 3-fluoro-3-deoxy-sugar (and/or downstream metabolites of the polyol and acid) in internal organ(s) of said subject and/or whole body, to assess a disease state, e.g., one that is associated with a pattern of abnormal activity of oxidizing and/or reducing enzymes, which are aldehyde dehydrogenase and aldose reductase, respectively, for example, assessing and monitoring a tumor showing increased activity of one or both oxidizing and reducing enzymes.

The invention also relates to a method of treating cancer in a patient, wherein the cancer is determined to be associated with elevated activity of at least one of aldehyde dehydrogenase and aldose reductase, comprising the steps of:
identifying generation, localization and/or distribution of one or more fluorinated compounds by PET as explained above; wherein when the PET indicates a localized disease, treating the patient by surgery and/or radiotherapy;
when the PET indicates a locally-advanced disease, treating the patient by surgery and/or radiotherapy; and
when the PET indicates an advanced, spread (metastatic) disease state, treating the patient by systemic administration of one or more active agents.

The PET procedure may be aided by administration of selective enzyme inhibitors, to enable determination of the 3FD-sugar-metabolizing enzyme(s) involved in the metabolic process detected by the PET.

The life-time of ¹⁸F is ~110 minutes. To prepare 3-¹⁸F-3-deoxy-D-galactose or 3-¹⁸F-3-deoxy-D-glucose, the radioisotope ¹⁸F is produced by a cyclotron, for example, via the ¹⁸O(p,n)¹⁸F reaction or the ¹⁶O(p,n)¹⁸F reaction as described by Knust et al. [Int. J. Appl. Radiat. Isot. 34 1627 (1983) and Appl. Radiat. Isot. 37, 853 (1986). ¹⁸F is incorporated into the compound via an appropriate synthetic pathway. Specifically, a fluorinated sulfonate, such as the triflate group (—O—SO₂—CF₃, —OTf) attached to a five or six-membered ring can be displaced by a fluoride ion, and therefore triflate derivatives are useful as precursors of the 3-¹⁸F-labeled sugars.

The reaction steps that follow the displacement of the triflate group by ¹⁸F consist of removal of alcohol protection groups, that were previously introduced into the sugar, to restore the hydroxyl groups of the sugar (other than of course at the C-3 position). Protecting groups are usually removed by acid hydrolysis or by base hydrolysis. Cyclotron-produced radiopharmaceutical set-ups may not have the tools to perform both types of hydrolysis. It is therefore desirable to provide synthetic pathways producing chemical precursors of the sugars that are amenable to deprotection under either acidic or basic conditions.

An example of an acid-cleavable protecting group is the ketal group. Thus, the ketal-protected 3-triflate-3-deoxy gulofuranose of formula IA (a precursor of 3-[¹⁸F]FDGal) and the ketal-protected 3-triflate-3-deoxy allofuranose of Formula IB (a precursor of 3-[¹⁸F]FDG), having the structures shown below:

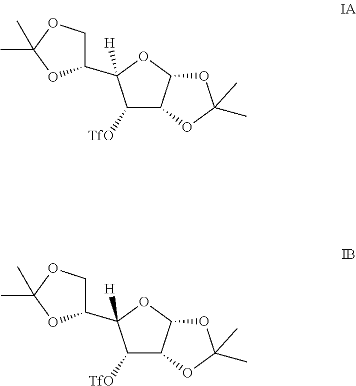

can be used in cyclotron-produced radiopharmaceutical setup equipped with acid hydrolysis.

These precursors can be prepared by published procedures (e.g., Hall et al. Carbohydrate Res 47: 299-305 (1976) and Tewson et al., J. Nuc. Med 19: 1339-1345 (1978)), showing a multistep synthesis, with addition of triflic anhydride (Tf₂O) to a cooled solution of 1,2:5,6-Di-O-isopropylidene-ca-D-allofuranose, or 1,2:5,6-Di-O-isopropylidene-α-D-gulofuranose, in pyridine; the triflate precursors of Formulas 1A and 1B are recovered.

Next, the triflate group is displaced by ¹⁸F, supplied in the form of Cs¹⁸F as shown by Tewson (supra) or by other sources, with the help of commonly used [¹⁸F] kryptofix procedure. Finally, removal of the di-isopropylidene groups is by acid hydrolysis as shown in the multistep reaction schemes depicted below, or with boron trichloride as described by Tewson (supra).

### Preparation of 3-¹⁸F-3-deoxy-D-galactose

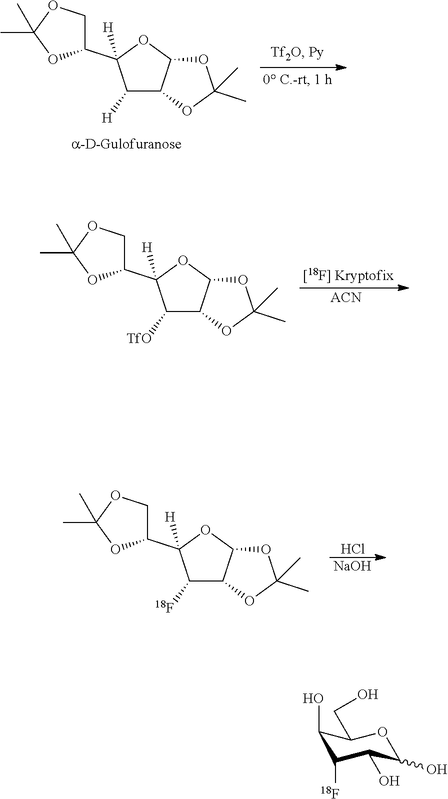

### Preparation of 3-¹⁸F-3-deoxy-D-glucose

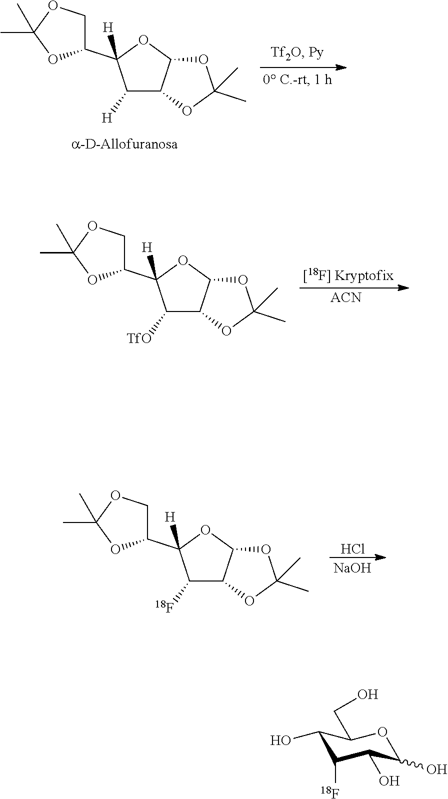

An example of a base-cleavable protecting group is the acetate. Thus, the acetate-protected 3-triflate-3-deoxy gulopyranose of Formula IIA (a precursor of 3-[¹⁸F]FDGal) and the acetate-protected 3-triflate-3-deoxy allopyranose of Formula IIB (a precursor of 3-[¹⁸F]FDG), having the structures shown below:

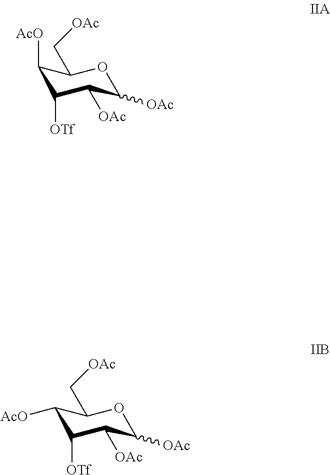

can be used in a cyclotron-produced radiopharmaceutical setup equipped with base hydrolysis.

Thus, the invention relates to a process for preparing 3-¹⁸F-3-deoxy-D-galactose, comprising the steps of:
A) providing acetate-protected 3-triflate-3-deoxy gulopyranose:

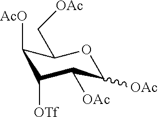

B) displacing the triflate group at position 3 of the acetate-protected 3-triflate gulopyranose by ¹⁸F:

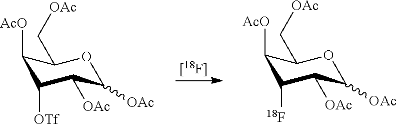

C) removing the acetate protecting groups by base hydrolysis:

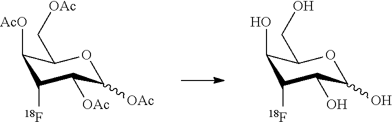

The multistep reaction shown below, which forms a separate aspect of the invention, provides the acetate-protected 3-triflate gulopyranose and the acetate-protected 3-triflate-allopyranose—the key precursors supplied to a cyclotron-based radiopharmaceuticals production apparatus.

The multistep reaction starts from the commercially available 1,2:5,6-Di-O-isopropylidene-α-D-gulofuranose/allofuranose:

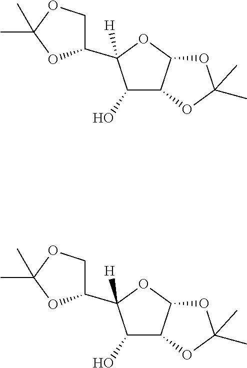

and enables the introduction of multiple acetate groups into the sugar system, to afford the acetate-protected 3-triflate glucopyranose/allopyranose with enrichment of the six-membered sugar along the synthesis (selectivity of pyranose over furanose). The synthesis that starts from 1,2:5,6-Di-O-isopropylidene-α-D-gulofuranose is depicted below for the purpose of illustration, but it should be understood that the corresponding synthesis utilizing 1,2:5,6-Di-O-isopropylidene-α-D-allofuranose as the starting material is also provided by the invention:

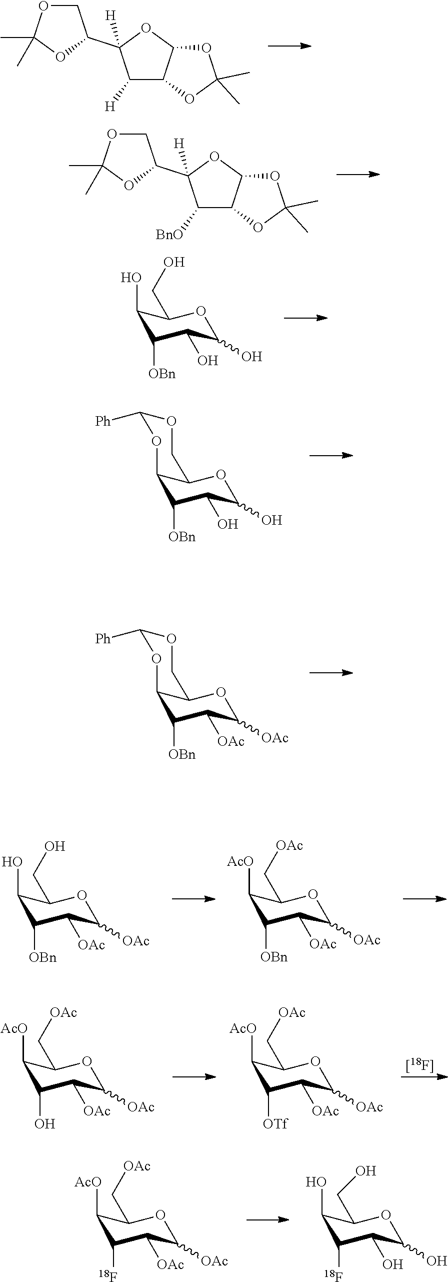

The synthesis starts with benzyl protection of the alcohol functionality of 1,2:5,6-Di-O-isopropylidene-α-D-gulofuranose with benzyl halide, e.g., benzyl bromide, in the presence of a base, e.g., a strong base such as sodium hydride:

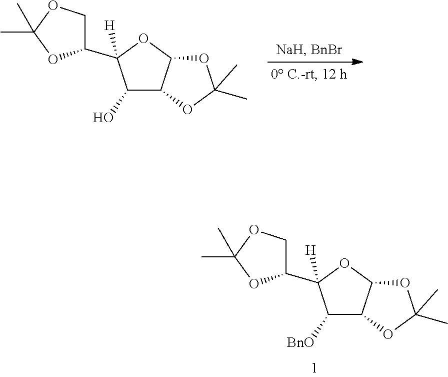

The benzyl protection reaction takes place in aprotic solvent such as dimethylformamide under cooling. The benzyl ether compound (1) is recovered from the reaction mixture in a solid form.

The next step is the cleavage of the 0-isopropylidene protection group:

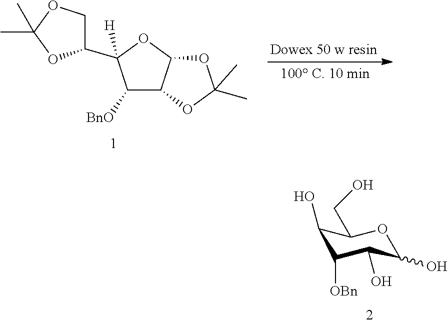

This deprotection step can be achieved by dissolving the benzyl ether compound (1) in water (or aqueous/organic water medium) in acidic environment, e.g., with the help of ion exchange resin in the hydrogen form. The product (2) that is recovered from the reaction mixture in a solid form consists in fact of a mixture of five and six-membered rings. The isolated solid proceeds without further purification to the next step.

Difficulties encountered with selective separation of the six-membered ring and direct simultaneous acetylation at positions 1, 2, 5 and 6 to form the tetra acetate derivative were solved by first passing via an acetal protected-intermediate, e.g. a benzaldehyde dimethyl acetal protected-intermediate 3:

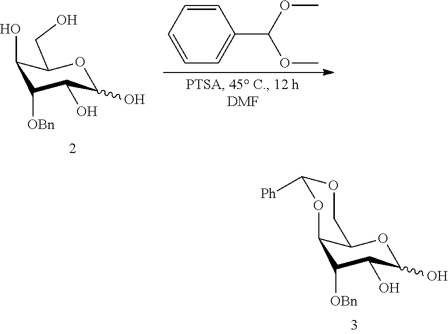

The protection reaction of 2-3 is carried out in an organic solvent, e.g., DMF, in the presence of p-Toluene sulfonic acid, e.g., at slightly elevated temperature over a few hours. The reaction mixture is worked up to recover a crude residue, which can be purified by column chromatography to afford compound 3 as a white solid.

The benzaldehyde dimethyl acetal protected-intermediate 3 is amenable to acetylation reaction at the hydroxyl-occupied positions:

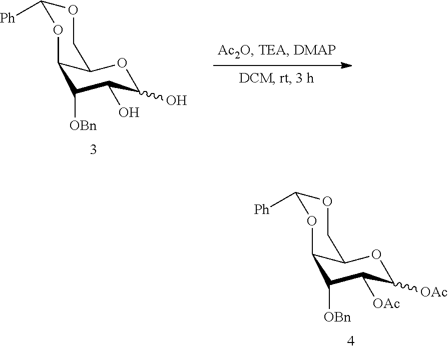

Acetylation of 3 with acetic anhydride is carried out in the presence of 4-(dimethylamino)pyridine and optionally an auxiliary base, e.g., triethylamine in an organic solvent, e.g., halogenated hydrocarbon such as dichloromethane. The reaction mixture is worked up to isolate 4 in a solid form.

Deprotection of 4, to cleave the benzaldehyde dimethyl acetal protecting group and free the corresponding hydroxyls:

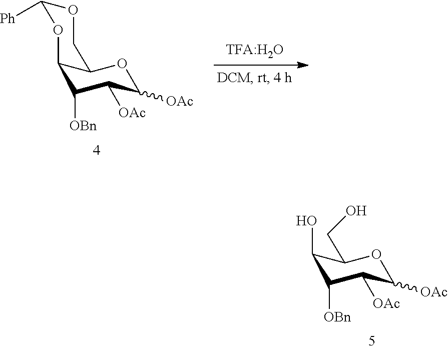

is catalyzed by an acid, e.g., a strong organic acid such as aqueous trifluoroacetic acid in an organic solvent such as dichloromethane. Upon completion of the deprotection reaction and neutralization with a base, followed by separation into organic and aqueous phases and concentration of the organic phase, compound 5 is recovered as a colorless oil.

The acetylation to afford the tetra-acetate 6

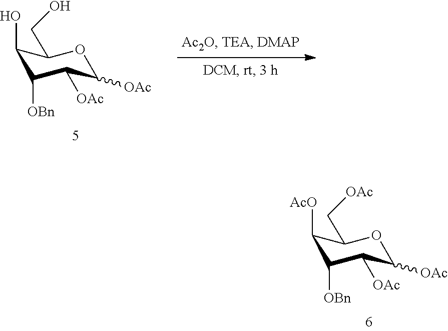

is accomplished with acetic anhydride as described above for the 3-4 reaction.

The next step consists of benzyl deprotection by catalytic hydrogenolysis (e.g., in ethyl ethanoate) to restore the 3-OH functionality of the sugar:

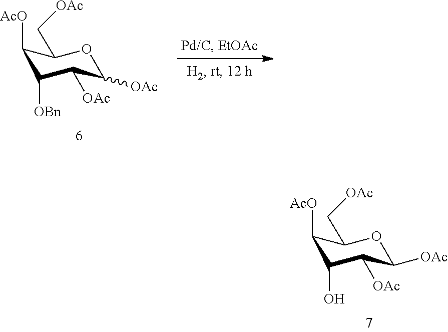

To this end, compound 6 is dissolved in EtOAc and the reaction mixture is stirred in the presence of a suitable catalyst such as Pd/C under an H₂ atmosphere. Upon separation of the catalyst by filtration, concentration under reduced pressure and purification by column chromatography, compound 7 is obtained as a white solid β-isomer.

With the addition of triflic anhydride (Tf₂O) to a cooled solution of 7 (e.g., in dichloromethane) in the presence of pyridine:

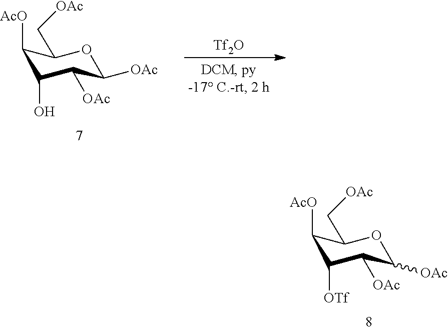

the key precursor 8 is formed and isolated from the reaction mixture. Precursor 8 constitutes another aspect of the invention. The precursor 8 is supplied to a cyclotron-based radiopharmaceuticals production facility as described above, where the triflate group is displaced by ¹⁸F, with the aid of potassium carbonate and Kryptoflix® 222, followed by base hydrolysis. See also a procedure described by Zhou, D., Chu, W., Xu, J. et al. [¹⁸F]Tosyl fluoride as a versatile [¹⁸F]fluoride source for the preparation of ¹⁸F-labeled radiopharmaceuticals. Sci Rep 13, 3182 (2023). https://doi.org/10.1038/s41598-023-30200-2.

For the use of a 3-¹⁸F-3-deoxy-sugar as radiotracer in PET scans, protocols akin to the current protocols of 2-[¹⁸F]FDG can be applied. For example, 3-¹⁸F-3-deoxy-D-galactose or 3-¹⁸F-3-deoxy-D-glucose can be delivered to the patient parenterally, e.g., by intravenous injection. For example, 3-¹⁸F-3-deoxy-D-galactose can be supplied in a bulk form by the manufacturer and an injectable composition is prepared prior to use by dissolving an appropriate amount of 3-¹⁸F-3-deoxy-D-galactose in a saline solution (0.9% sodium chloride solution). The amount of the ¹⁸F-labeled sugar added to the solution is calculated considering the decay of the isotope and the prescribed dose to be delivered to the patient, e.g., 0.1 mCi/kg body weight. Upon sterilization, an injectable solution is obtained. 3-¹⁸F-3-deoxy-D-galactose can also be supplied in a ready-to-use form, e.g., an injectable solution packaged in a multiple-dose glass vial or filled syringes, with 20 to 200 mCi/ml of 3-¹⁸F-3-deoxy-D-galactose in a saline isotonic solution that is optionally buffered (phosphate, histidine or citrate buffer).

The PET scan proceeds in a similar manner to 2-[¹⁸F]FDG. Acceptable dosage of injected activity is from 0.05 to 0.5 mCi/kg body, e.g., around 0.1 mCi/kg body. Whole-body PET is acquired ~1 h after intravenous injection of the radiotracer. For example, dynamic acquisition may be performed between t=0 and 30 min, then switching to static acquisition at 15 min intervals up to 5 hours; or static acquisition at any time between 40 min and 5 hours. Image acquisition length changes between different PET systems but should be between 3-5 minutes and 15 minutes for most systems.

Accordingly, the invention further provides 3-¹⁸F-3-deoxy-sugar (e.g., 3-¹⁸F-3-deoxy-D-galactose, 3-¹⁸F-3-deoxy-D-xylose or 3-¹⁸F-3-deoxy-D-glucose) for use a radiotracer in PET for detection and/or monitoring of a disease that exhibits abnormal activity pattern of oxidizing and/or reducing enzymes. For example, diseases exhibiting abnormal activity pattern of both types of enzymes (oxidizing and reducing enzymes).

The diagnostic test provided by the invention based on 3-¹⁸F-3-deoxy-sugar PET is well suited for the assessment, diagnosis and/or monitoring of the disease states listed above, e.g. metabolic diseases and cancer.

A parenterally administrable composition comprising 3-¹⁸F-3-deoxy-sugar and a carrier for use in PET for detecting and/or monitoring of a disease that exhibits abnormal pattern of activity of oxidizing and/or reducing enzymes forms another aspect of the invention, e.g., an injectable composition comprising an isotonic saline, sterile, optionally buffered solution of 3-¹⁸F-3-deoxy-D-galactose for use in PET. Suitable buffers were mentioned above.

### BRIEF DESCRIPTION OF THE DRAWINGS

FIG. 1 shows the interaction of 3FDG and 3FDGal with endogenous enzymes, revealing cellular oxidation or reduction.

FIGS. 2A and 2B show schemes of phantom experiments conducted with tubes containing the 3FD-compounds (2A—the sugar 3FDG and its metabolites 3FDS and 3FDGA; 2B—the sugar 3FDGal and its metabolites 3FDGtol and 3FDGalA), verifying the feasibility of spectrally differentiating the ¹⁹F-MR signals of the sugars and their metabolites, and the ability to spatially map the sugar and metabolites with a single mCSSI-based FMI acquisition.

FIG. 3 shows the in-vivo metabolism of systemically administered 3FDG to mice.

FIG. 4 shows the in-vivo metabolism of systemically administered 3FDGal to mice.

FIG. 5 is localized ¹⁹F-MR spectroscopy of 3FDGal, overlaid on 1H-MRI data.

FIG. 6 shows the in-vivo metabolite yield of 3FDGal, concurrently reporting the spatial distribution of sugar oxidizing and reducing enzymes, generating 3FDGalA and 3FDGtol, respectively.

FIG. 7 shows the results of in-vitro incubation studies based on 3FDGal transport into different cell lines, and the metabolites produced in the cells.

FIG. 8 shows a ¹⁹F MR-generated map of athymic nude mice upon administration of 3FDGal to mice that was subcutaneously injected with A549 and LNCaP cells to induce proliferating subcutaneous tumors. Both 3FDGalA and 3FDGtol formation by A549 tumors and no 3FDGal metabolism by LNCaP tumors are observed.

FIG. 9 shows the flowchart (flow diagram) of the synthetic process of preparation of 3FDGal from 3-Deoxy-3-fluoro-1,2:5,6-bis-O-(1-methylethylidene)-ca-D-galactofuranose in a QMA cartridge.

FIG. 10 shows the ¹⁹F-NMR spectra of 3FDGal synthesis in the QMA cartridge.

FIGS. 11A-11N: Identifying the human enzymes driving the metabolic schemes of 3FDG and 3FDGal. FIG. 11A-FIG. 11B show schematic overview of the experimental design. HEK-293 cells were transfected with expression vectors (one vector at a time) that encode for the overexpression of different enzymes, to assess their potential to intracellularly reduce or oxidize 3FDG or 3FDGal (FIG. 11A). The cells were then incubated with either 3FDG or 3FDGal, followed by ¹⁹F-NMR of the intracellular (IC) content (FIG. 11B) (n=3 biologically independent experiments in all groups). FIG. 11C shows a Western blot analysis of the transfected cells confirming the successful expression of the examined proteins. FIG. 11D-FIG. 11E show representative high-resolution ¹⁹F{¹H}-NMR spectra of the IC content acquired in one control (HEK-293^{NT}) and two experimental conditions (HEK-293^{AKR1B1} and HEK-293^{ALDH1A1}) following incubation with 3FDG (FIG. 11D) or 3FDGal (FIG. 11E). ¹⁹F-NMR spectra from HEK-293^{NT} show endogenous generation of 3FDS (but not 3FDGA) from 3FDG, and 3FDGtol (but not 3FDGalA) from 3FDGal. Spectra from HEK-293^{AKR1B1} show enhanced generation of 3FDS (from 3FDG) and 3FDGtol (from 3FDGal). Spectra from HEK-293^{ALDH1A1} show induced generation of 3FDGA (from 3FDG) and 3FDGalA (from 3FDGal). FIG. 11F-FIG. 11G show quantification of ¹⁹F-NMR SNR values calculated in all conditions at the relevant chemical shifts: δ=−212.4 (3FDS) and δ=−208.4 (3FDGA) in cells incubated with 3FDG (11F), and δ=−209.9 ppm (3FDGtol) and δ=−205.1 ppm (3FDGalA) in cells incubated with 3FDGal (FIG. 11G). FIG. 11H shows that after co-transfecting HEK-293 cells with AKR1B1 and SORD (schematic presentation on the top), Western blot analysis confirmed the successful expression of both proteins (bottom). FIG. 11I-FIG. 11L show representative high-resolution ¹⁹F{¹H}-NMR spectra of the IC content acquired in HEK-293^{AKR1B1SORD} cells show 3FDS and 3-fluoro-3-deoxy-fructose (3FDF) generation following incubation with 3FDG (FIG. 11I), versus 3FDGtol-only generation following incubation with 3FDGal (FIG. 11K). Quantification of ¹⁹F-NMR SNR values calculated at δ=−212.4 (3FDS) and δ=−207.7 (3FDF) in HEK-293^{AKR1B1} versus HEK-293^{AKR1B1+SORD} cells incubated with 3FDG (FIG. 11J), and at δ=−209.9 ppm (3FDGtol) in HEK-293^{AKR1B1} versus HEK^{AKR1B1+SORD} cells incubated with 3FDGal (FIG. 11L). FIG. 11M-FIG. 11N show the intracellular trapping mechanism for 3FDG (FIG. 11M) versus 3FDGal (FIG. 11N). SNR, signal-to-noise ratio. Data presented as mean values±s.d.

FIGS. 12A-12G show recording 3FDGal metabolism with ¹⁹F-NMR enables fingerprinting of cancer cells. FIG. 12A shows schematic presentation of the in vitro incubation experiment. Different human cancer cell lines were incubated with 3FDGal (6×10⁶ cells; 5 mM 3FDGal; 4 hrs; n=3 biologically independent experiments per cell line). Then, the intracellular (IC) content of the cells was analyzed on ¹⁹F-NMR, GC-MS, and Western blot. FIG. 12B shows representative high-resolution ¹⁹F{¹H}-NMR spectra of the IC content for each of the assessed cell lines. FIG. 12C-FIG. 12D show quantified SNR values calculated for the ¹⁹F-NMR peaks of 3FDGalA at δ=−205.1 ppm (top) and of 3FDGtol at δ=−209.9 ppm (bottom), represented per cell line on bar plots (FIG. 12C) and on a heatmap that illustrates the dual metabolic color-coded ¹⁹F-NMR fingerprints of the cells (FIG. 12D). FIG. 12E shows 3FDGalA (top) and 3FDGtol (bottom) concentrations, as determined by analytical GC-MS, presented per cell line on bar plots. FIG. 12F shows concentrations of 3FDGalA (top) and 3FDGtol (bottom) as a function of the SNR values calculated for the ¹⁹F-NMR peaks of 3FDGalA at δ=−205.1 ppm (top) and 3FDGtol at δ=−209.9 ppm (bottom) in all analyzed IC samples. A statistically significant excellent correlation was found between ¹⁹F-NMR-based and GC-MS-based values in both plots (Pearson's r=0.99, p-value <0.001). FIG. 12G shows Western blot of the analyzed cancer cells for ALDH1A1 and AKR1B1 expression. GC-MS, Gas chromatography-mass spectrometry. Data are presented as mean values±s.d.

FIGS. 13A-13C show imaging data of a representative mouse: ¹H-MRI anatomic axial slice at the level of the two tumors (13A) and ¹⁹F-MRI-based axial 3D-FMI-2 data from the corresponding axial location, overlaid on anatomical ¹H-MRI data (13A, right and center panels), which indicate 3FDGalA and 3FDGtol generation in the A549-derived tumor but not in the LNCaP-CL1-derived tumor. FIG. 13B show comparison of quantitative image-based parameter (SNRmax, TMV, and TTM) values as calculated in the A549- versus the LNCaP-CL1-derived tumors (data from VOIs covering the tumors at 3D-FMI-2). FIG. 13C show H&E staining and immunostaining (anti-ALDH1A1 and anti-AKR1B1) of the tumors studied in vivo.

FIGS. 14A-14G. FIG. 14A shows a heatmap that illustrates the predicted proteomic-based dual metabolic color-coded ¹⁹F-NMR fingerprints of all NSCLC cell lines included in the NCI-60 panel. FIG. 14B shows representative high-resolution ¹⁹F{¹H}-NMR spectra of the IC content for NCIH460 (top) and NCIH522 (bottom) cells after their incubation with 3FDGal. FIG. 14C shows a quantified SNR values calculated for the ¹⁹F-NMR peaks of 3FDGalA at δ=−205.1 ppm and of 3FDGtol at δ=−209.9 ppm. FIG. 14D schematically show NCIH522 and NCIH460 cells were subcutaneously injected (right and left, respectively) to induce a bilateral xenograft mouse model. FIG. 14E-FIG. 14G show 3D-FMI-2 data overlaid on anatomical 1H-MRI data at the level of the two induced tumors indicate 3FDGalA^{high}/3FDGtol^{low} and 3FDGalA^{low}/3FDGtol^{high} imaging phenotypes in NCIH522- and NCIH460-derived tumors, respectively, mirroring their ALDH1A1^{high}/AKR1B^{low} and ALDH1A1^{low}/AKR1B1^{high} proteomic phenotypes, respectively. SNRmax, maximal signal-to-noise ratio. TMV, tissue metabolite volume. TTM, total tissue metabolite. VOI, volume-of-interest. H&E, hematoxylin and eosin. NSCLC, non-small cell lung cancer. NCI-60, National Cancer Institute-60.

FIG. 15 shows a heatmap that illustrates the predicted proteomic-based dual metabolic color-coded ¹⁹F-NMR fingerprints of all RCC cell lines included in the NCI-60 panel.

FIGS. 16A-16B show a schematic presentation of the experimental procedure (FIG. 16A) and a two time points FMI of a representative mouse (FIG. 16B).

FIGS. 17A-17C show a schematic presentation of the experimental procedure (FIG. 17A) and detection of the xenografts (17B), visualizing the expected FMI phenotype (ACHN: high AKR1B1 expression, mild ALDH1A1 expression based on both proteomic and Western blot (WB) data. Quantification is shown in FIG. 17C.

FIGS. 18A-18B show a schematic presentation of the experimental procedure (FIG. 18A) and demonstrates also with 3FDG the ability to visualize uptake of 3FDG (green) followed by reduction d/t high AKR1B1 activity (conversion to 3FDS, red).

FIGS. 19A-19C show a schematic presentation of the experimental procedure (FIG. 19A) and fast FMI with 3FDGal, reducing acquisition time to 20 minute only per step, which is graphically shown in FIG. 19C.

FIGS. 20A-20D show recording of 3FDGal metabolism with ¹⁹F-NMR in two different cancer cells used to induce tumors in the brain. FIG. 20A shows a ¹⁹F-NMR spectrum of the intracellular (IC) content of A549 cells after incubation with 3FDGal. FIG. 20B shows a quantified SNR values calculated for the ¹⁹F-NMR peaks of 3FDGalA (−205.1 ppm) and of 3FDGtol (−209.9 ppm) as obtained for A549 cells (N=3). FIG. 20C shows a ¹⁹F-NMR spectrum of the intracellular (IC) content of TBDRG-05MG cells after incubation with 3FDGal. FIG. 20D shows quantified SNR values calculated for the ¹⁹F-NMR peaks of 3FDGalA (−205.1 ppm) and of 3FDGtol (−209.9 ppm) as obtained for TBDRG-05MG cells (N=3).

FIGS. 21A-21C shows a three-dimensional Fluorine Metabolic Imaging (3D-FMI) of the brain. Healthy mouse (top row, FIG. 21A) and mice inoculated with two types of tumors in their brains were studied using 3D-FMI 2 hours post-3FDGal injection. Shown are, from left to right, 4 different MRI data sets for each mouse representing an axial view of a single selected slice of the brain, anatomical ¹H-MRI, 3FDGal map (overlayed on the corresponding 1H-MRI data), 3FDGalA map (overlayed on the corresponding 1H-MRI data), and 3FDGtol map (overlayed on the corresponding 1H-MRI data). After intraperitoneal injection of 3FDGal (0.6 g/kg) at t=0, axial mCSSI-based 3D-FMI was acquired to simultaneously map 3FDGal, 3FDGalA, and 3FDGtol. Slices of 3 mm thickness and an in-plane resolution of 2 mm×2 mm were acquired. Top row: healthy mouse. FIG. 21B (middle row): mouse inoculated with 200,000 A549 (non-small cell lung cancer) cells expressing high levels of ALDH1A1 and AKR1B1, thus producing both 3FDGalA (shown as gold color) and 3FDGtol (shown as magenta) from the introduced 3FDGal in the tumor region. FIG. 21C (Bottom row): mouse inoculated with 200,000 DBTRG-05MG (glioma) cells expressing high levels of AKR1B1, thus producing 3FDGtol (shown as magenta) from the introduced 3FDGal in the tumor region. Data was acquired on a 15.2 T MRI scanner.

FIG. 22 shows two-dimensional Fluorine Metabolic Imaging (2D-FMI) of the brain following the injection of 3FDG. 3FDG (0.6 g/kg) was intraperitoneally administered to healthy C57BL/6 male mice, and coronal mCSSI-based 2D-FMI was performed repeatedly, starting at t=60 min post-injection to map 3FDG (FIG. 22A, green colored overlay on ¹H-MRI of the corresponding brain slice), 3FDGA (FIG. 22B, not shown in the brain), and 3FDS (FIG. 22C, red colored overlay on ¹H-MRI of the corresponding brain slice) simultaneously and noninvasively to visualize the real-time in vivo metabolism of 3FDG. The in vivo imaging data highlight the early, widespread uptake of the injected 3FDG, followed by metabolic reduction to 3FDGS, revealing their spatial distributions. Note that 3FDGA, the oxidized metabolite (i.e., 3FDGA) of 3FDG, was not found in the brain of healthy mice. MRI experiments were done on a 15.2 T MRI scanner with a 23 mm 1H/19F RF volume coil. A 20 mm single slice with in-plane resolution of 2 mm×2 mm was acquired at each 2D-FMI time point.

FIGS. 23A-23C shows a two-dimensional Fluorine Metabolic Imaging (2D-FMI) of the brain following the injection of 3FDGal. 3FDGal (0.6 g/kg) was intraperitoneally administered to healthy C57BL/6 male mice, and coronal mCSSI-based 2D-FMI was performed repeatedly, starting at t=60 min post-injection to map 3FDGal (FIG. 23A, green colored overlay on ¹H-MRI of the corresponding brain slice), 3FDGalA (FIG. 23B, gold colored overlay on ¹H-MRI of the corresponding brain slice), and 3FDGtol (FIG. 23C, magenta colored overlay on 1H-MRI of the corresponding brain slice) simultaneously and noninvasively to visualize the real-time in vivo metabolism of 3FDGal. The in vivo imaging data highlight the early, widespread uptake of the injected 3FDGal, followed by its disappearance and metabolic oxidation to 3FDGalA (first time point in the olfactory bulb ROI) and reduction to 3FDGtol, revealing their spatial distributions. MRI experiments were done on a 15.2 T MRI scanner with a 23 mm 1H/19F RF volume coil. A 20 mm single slice with in-plane resolution of 2 mm×2 mm was acquired at each 2D-FMI time point.

FIGS. 24A-24B show in vivo biodistribution of 3FDXyl metabolites in healthy mouse. FIG. 24A shows a chemical structures and ¹⁹F-NMR spectra of 3FDXyl and its redox derivatives (3FDXA, 3FDXtol). FIG. 24B shows a preliminary in vivo FMI after administration of 3FDXyl. The non-localized ¹⁹F-NMR acquired after probe injection shows the longitudinal transformation of 3FDXyl to its redox metabolites, and the FMI maps show the biodistribution of 3FDXtol (magenta) and 3FDXylA (gold).

FIG. 25 shows fFMI acquisition protocol improving both temporal resolution (reducing time acquisition from 80 to 20 minutes per step) and spatial resolution (decreasing voxel size from 2×2×3 mm to 1.5×1.5×3 mm, a ~44% reduction). These enhancements were achieved through bandwidth optimization: narrowing the receiver BW from 50 kHz to 10 kHz and reducing the excitation/refocusing BW from 2 kHz to 1 kHz. Data quantification (for LNCaP-CL1 tumor in the upper row and A549 tumor in the lower row) is based on a phantom tube containing known concentrations of 3FDGalA and 3FDGtol.

FIG. 26 is rate versus substrate concentration graph showing AKR1B1 enzyme kinetics with various sugar substrates.

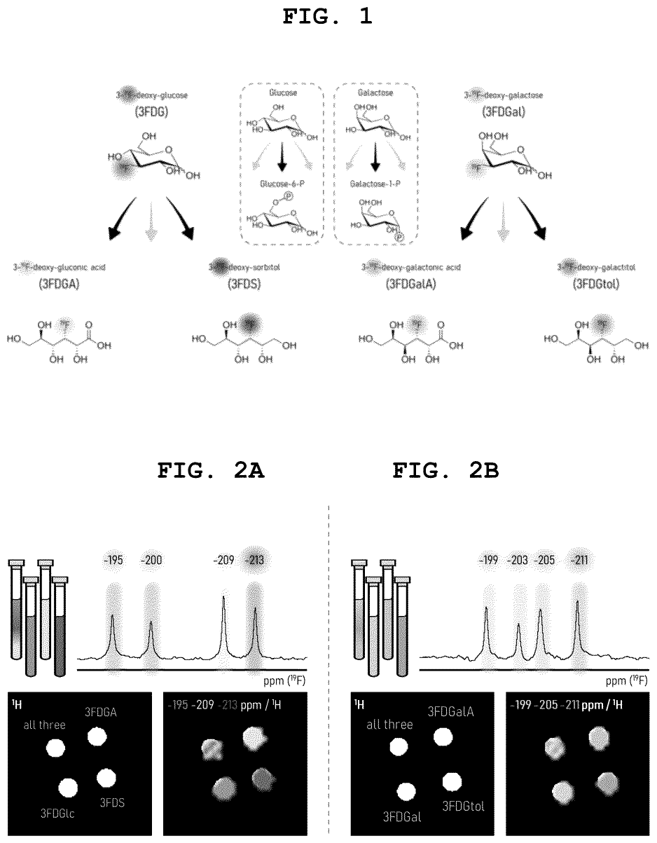

### EXAMPLES

### Materials

3FDG and 3FDGal were purchased from Omicron Biochemicals, Inc., USA. 1,2:5,6-Di-O-isopropylidene-α-D-allofuranose and 1,2:5,6-Di-O-isopropylidene-α-D-gulofuranose were purchased from Aaron Chemicals LLC. (https://www.aaronchem.com/). The ultrapure water used in all experiments was obtained using a Milli-Q apparatus.

### Methods and Instrumentation

### Nuclear Magnetic Resonance (NMR)

All NMR measurements were performed on a Bruker AVANCE NEO-400 MHz spectrometer (9.4T), driven by TopSpin 4.3.0 (Bruker). All samples contained 10% D₂O as a locking substance. ¹⁹F{¹H} NMR experiments were performed after tuning the RF coils to both ¹⁹F and ¹H frequencies and following an automatic shimming procedure. The following parameters were used for acquisition: Receiver gain (RG)=101, acquisition time (AQ)=884.7360 msec, and repetition time (D1)=5 sec. ¹⁹F{¹H} NMR measurements of both the intracellular (IC) and extracellular (EC) samples were done after setting the number of scans to 225 and 25, respectively [resulting in a total scan time=~22.5/~2.5 min]. SNR calculations were performed on the processed data using the sinocal command (noise was calculated in all samples in a 1 ppm segment from −220 to −221 ppm).

### Magnetic Resonance Imaging (MRI)

### General Procedure

All MRI data were acquired on a Bruker AVIII-400WB (9.4T) wide-bore vertical spectrometer, equipped with a Micro 2.5 imaging probe with actively shielded gradients, using the MICWB40 RES 400 ¹H/¹⁹F 040/025 LLTR RF volume coil (Bruker, Model no. T121569, 25 mm diameter) and driven by ParaVision 360 (Bruker). The spectrometer operated at frequencies of 400.21 MHz for ¹H and 376.54 MHz for ¹⁹F-MR measurements.

### In-Vivo Imaging Setup

Mice were anaesthetized with isoflurane (ISOFLURANE, USP 100%, TERRELL™, PIRAMAL CRITICAL CARE, USA), delivered in 50%/50% oxygen/nitrogen gas. Induction of anesthesia was given in a chamber with 3-5% isoflurane. Upon deep anesthesia, the ¹⁹F-labeled probe was administered intravenously via retro-orbital injection (15 mg probe in 70 μl PBS, ~0.6 g/kg). The time of probe injection was defined as t=0 in all cases. At that point and until the end of imaging, the mice were positioned in a dedicated imaging bed (MICWB40 M.BED 025 CPL, Bruker), and anesthesia was delivered through a nose cone, with maintenance isoflurane level set to 1-2%. Respiration was monitored by a M1025 system (SA Instruments), using a pneumatic pillow positioned at the animal's chest. Between t=0 and t=30 min, the animal was fixed to the mini-imaging bed and inserted into the scanner, followed by initial MRI adjustments done to ensure mice positioning (anatomic coverage of animal's chest and abdomen), tune and match the ¹H/¹⁹F frequencies, set the ¹H reference power (generally calculated automatically between 0.14 and 0.21 W; the same value was used as the reference power for ¹⁹F measurements as well), and complete a BC map-based shimming. Upon completion of MRI adjustments, at the end of the mentioned time frame and right before the first FMI step, 1H-based anatomic data and non-localizing ¹⁹F-NMR data were recorded. The entire imaging session took between 3.5 and 5 h. After imaging, all animals recovered from anesthesia within minutes.

¹H Anatomic images

To correlate Fluorine Metabolic Imaging (FMI) data with morphological images, Bruker's Rapid Acquisition with Relaxation Enhancement (RARE) sequence was used as the measuring method, acquiring ¹H-based anatomic data with the following parameters: TE=8 ms; TR=6600 ms; RARE factor=8; Averages=2 [Scan time ~3.5 m]. For 2D high-resolution axial imaging, 96 axial slices were acquired with a fat suppression module (matrix size=128×128; FOV=32 mm×32 mm; slice thickness 0.5 mm; in-plane resolution=0.25 mm×0.25 mm). For 2D coronal imaging, 32 coronal slices were acquired with a fat suppression module (matrix size=128×128; FOV=48 mm×32 mm; slice thickness=1 mm; in-plane resolution=0.375 mm×0.25 mm).

### Non-Localizing ¹⁹F NMR Data

To record time-specific spectral data summing the overall ¹⁹F-labeled materials contained within the FOV (the ¹⁹F-labeled administered probe and its downstream metabolites), Bruker's Single Pulse sequence was used. This way, at different time points along the imaging session (generally, at t≈30 m, in between FMI steps, and at the end of the session), non-localizing data revealed the differential of ¹⁹F-containing metabolite content, based on which it was possible to extract the exact chemical shifts of each ¹⁹F-NMR peak (in MHz), serving as the selective excitation frequencies set on the following FMI. The following parameters were used: TR 750 ms; Flip angle=60°; Averages=240; Scan time=3 min; FID of 2048 pts was acquired during 55 ms; working chemical shift ~206 ppm; spectral bandwidth ~98 ppm].

### Fluoro-Metabolic Imaging (FMI)

### General Procedure

In order to concurrently record multiple metabolic images, a ¹⁹F-multi chemical shift selective imaging (mCSSI) was applied, as described by [Flagel U., et al., “Multi-targeted ¹H/¹⁹F MRI unmasks specific danger patterns for emerging cardiovascular disorders”, Nature Communications, 12, 2021, 5847]. Briefly, mCSSI is a RARE sequence variant that replaces traditional RARE's slice selection with frequency selective excitation, applies narrow bandwidth excitations followed by phase encoding of the third dimension, and acquires the echo train with selective refocusing pulses. At the end of each cycle, gradient spoiling destroys the remaining transverse magnetization, and the entire procedure is repeated for the number of excited chemical shifts within a single TR. This way, setting n ¹⁹F chemical shifts during one FMI step, n matrices are acquired, each recording per-voxel signal intensity that corresponds to the spatial distribution of a specific frequency on the ¹⁹F NMR spectrum. Unless mentioned otherwise, we used the following parameters to acquire FMI data: TE=70 ms; TR=3000 ms; Echo spacing=4 ms; RARE factor=32; For 2D-FMI [Averages=1100, Scan time=55 min], a single coronal slice was acquired (matrix size=32×32; FOV=64 mm×64 mm; slice thickness=24 mm; in-plane resolution=2 mm×2 mm). For 3D-FMI [Averages=100, Scan time=1 h 20 min], 16 axial slices were acquired (matrix size=32×32; FOV=64 mm×64 mm; slice thickness=3 mm; in-plane resolution=2 mm×2 mm). For all FMI data presented, excitation frequencies were selected on the administered ¹⁹F-probe chemical shift (the center of the high-frequency beta anomer), as well as on the two peaks that correspond to the metabolites endogenously generated (the center of the peak containing the generated acid and the center of the generated polyol peak). The bandwidth of the excitation and refocusing pulses was set at 2000 Hz. As described, the exact selective frequencies were defined based on non-localizing ¹⁹F-NMR data acquired before each FMI step began.

### Data Visualization.

For FMI data acquired after 3FDG administration, green (RGB=0/255/0), yellow (RGB=255/255/0), and red (RGB=255/51/0) were respectively assigned to represent the signal recorded when the ¹⁹F-NMR peak excited on FMI contained that of 3FDG, 3FDGA, and 3FDS. Similarly, for FMI data acquired after 3FDGal administration, cyan (RGB=102/255/255), gold (RGB=255/204/0), and magenta (RGB=255/102/255) were respectively assigned to represent the signal recorded when the ¹⁹F-NMR peak excited on FMI was that of 3FDGal, 3FDGalA, and 3FDGtol. For standardization and possible (semi-) quantification/comparison between FMI slices, any FMI slice presented hereby (all slices, either axial or coronal had a matrix size of 32×32) is visualized as processed signal-to-noise ratio maps (SNR maps). To generate the SNR maps, we customized a Matlab (MathWorks) script to first calculate each slice's noise (defined as the standard deviation of the signal in a fixed 30×3 voxels ROI spatially located to the right of the imaging bed). Then, by defining an image window (SNRmin and SNRmax), and applying a linear scale bar of the color corresponding to the excited ¹⁹F-NMR peak, the ratio between the raw signal intensity in each voxel and the calculated noise of the slice could be visualized. For visualization of FMI data overlaid on anatomic data, we used ParaVision 360's “Image Fusion” application, assigning the ‘green’/‘yellow’/‘red’/‘cyan’/‘gold’/‘magenta’ color bar scales provided by the software's viewer.

### 2D FMI Quantification

To allow for quantitative comparison between 3FDG and 3FDGal endogenous metabolism, eight healthy C57BL/6 mice were injected with 3FDG (n=4) or 3FDGal (n=4), followed by in vivo imaging session consisting of five 2D-FMI steps. In all cases, SNR maps of 3FDGA and 3FDS (for those injected with 3FDG) or 3FDGalA and 3FDGtol (for those injected with 3FDGal) were generated as described per FMI step. An 11×7-voxels ROI centered at the animal's liver was drawn on all generated 3FDGA and 3FDGalA SNR maps. Similarly, a 5×4-voxels ROI centered at the animal's heart was drawn on all generated 3FDS and 3FDGtol SNR maps. For all 80 ROIs (8 mice, 5 FMI steps, 2 metabolites): the maximal SNR within the ROI was recorded as SNRmax; the number of voxels within the ROI containing an SNR value >4.5 was recorded as TMA (total metabolite area); The sum of SNR values stored within voxels in the ROI which store an SNR value >4.5 was recorded as TTM (total tissue metabolite) content.

### Preparation 1

### Synthesis of 3-fluoro-3-deoxysorbitol (3FDS)

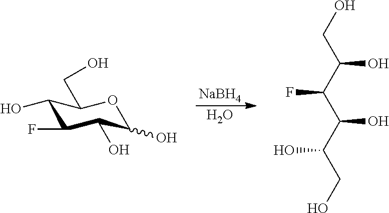

NaBH₄ (42 mg, 1.11 mmol) was added to the solution of 3-fluoro-3-deoxy-D-glucose (81 mg, 0.444 mmol) dissolved in water. TLC was used to monitor reaction completion, and the reaction mixture was loaded into a basic alumina column, water as eluent. The water-containing product was lyophilized to afford the 3-Deoxy-3-fluorosorbitol (52 mg, 63%) as a white solid.

### Preparation 2

### Synthesis of 3-fluoro-3-deoxy gluconic acid (3FDGA)

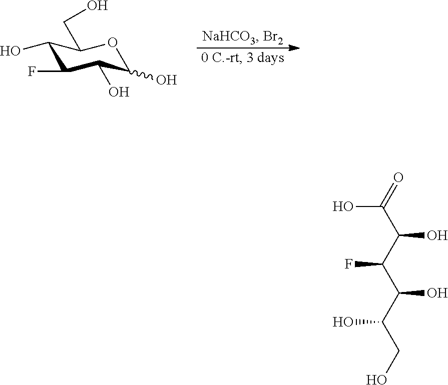

NaHCO₃ (55.3 mg, 0.658 mmol) was added to the solution of 3-fluoro-3-deoxy-D-glucose (60 mg, 0.329 mmol) and cooled the reaction mixture to 0° C. then Br₂ (18.6 μL, 0.362) was slowly added dropwise. The resulting solution was stirred for 48 hours. TLC was used to monitor reaction completion, and the reaction mixture was concentrated under reduced pressure. The crude residue was purified by column chromatography (CH₂Cl₂/MeOH, 4:1) to afford 3-deoxy-3-fluoro gluconic acid (80 mg, 71%) as a white solid.

### Preparation 3

### Synthesis of 3-fluoro-3-deoxy galactitol (3FDGtol)

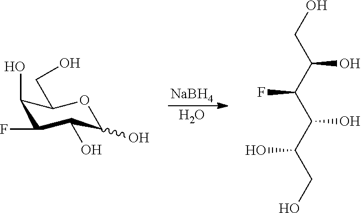

The procedure of Preparation 1 was followed, starting with 3-Fluoro-3-deoxy-D-galactose.

### Preparation 4

### Synthesis of 3-deoxy-3-fluoro galactonic acid (3FDGalA)

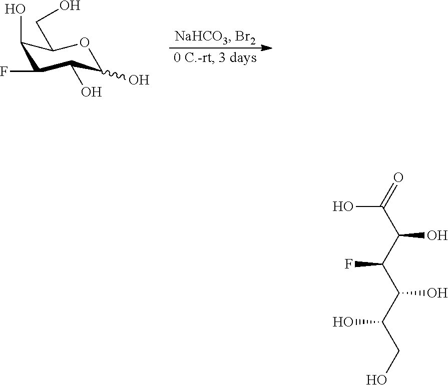

The procedure of Preparation 2 was followed, starting with 3-Fluoro-3-deoxy-D-galactose.

### Preparation 5

### Injectable Formulations of 3FDGal and 3FDG

The fluorinated sugar (30 g of either 3FDGal or 3FDG) was added to 300 ml of an aqueous solution that contains ~2.7 g of sodium chloride. The mixture was stirred at room temperature and sterilized to provide a clear, isotonic, sterile injectable solution.

The solution can be packed in a single-dose or multi-dose glass vial.

### Preparation 6

### Synthesis of ketal-protected 3-triflate-3-deoxy gulofuranose (a Precursor of 3-[¹⁸F]FDGal)

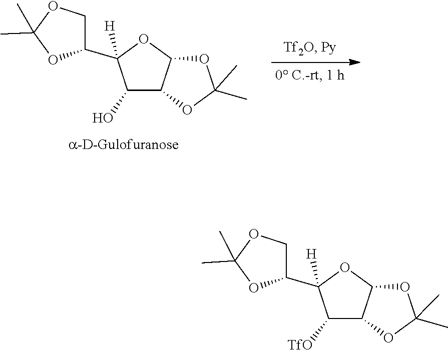

1,2:5,6-Di-O-isopropylidene-α-D-gulofuranose (75 mg, 0.288 mmol) was dissolved in pyridine and cooled to 0° C. and Tf₂O (57 μL, 0.345 mmol) was added dropwise over 10 minutes, and the resulting mixture was stirred for 1 h. TLC was used to monitor reaction completion and the RM was diluted with EtOAc and washed with 1N HCl solution (4×) and NaHCO₃ solution (3×). The combined organic layer was dried over Na₂SO₄, filtered, and concentrated under reduced pressure. The crude residue was purified by column chromatography (Ethyl acetate/Hexane, 1:6) to afford the ketal-protected 3-triflate-3-deoxy gulofuranose (80 mg, 71%) as a white colorless oil.

### Preparation 7

### Preparation of acetate-protected 3-triflate gulopyranose (a Precursor of 3-[¹⁸F]FDGal and 3-[¹⁸F]FDG)

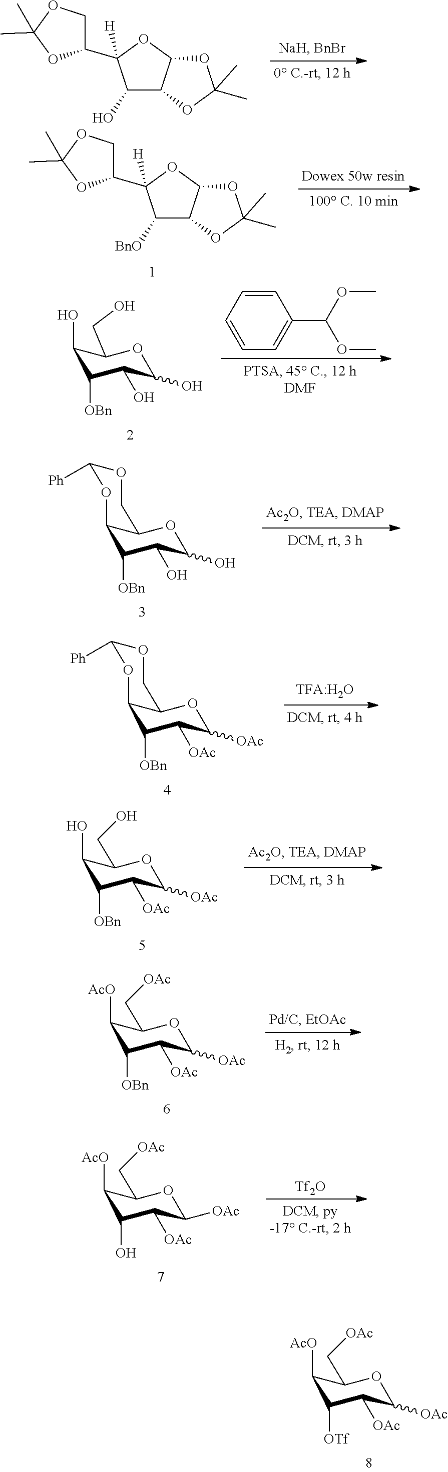

Synthesis of compound 1: 1,2:5,6-Di-O-isopropylidene-α-D-gulofuranose (106 mg, 0.407 mmol) was dissolved in anhydrous DMF (2 mL) and cooled to 0° C. NaH (19.5 mg, 0.814 mmol) was added, and after 10 minutes BnBr (72.6 μL, 0.61 mmol) was added dropwise over 10 minutes and the resulting mixture was stirred for 12 h. TLC was used to monitor reaction completion and the RM was diluted with ethyl acetate and washed with water and brine solution (3×) alternatively. The combined organic layer was dried over Na₂SO₄, filtered, and concentrated under reduced pressure. The crude residue was purified by column chromatography (Ethyl acetate/Hexane, 1:6) to afford compound 1 (115 mg, 80%) as a white solid.

Synthesis of compound 2: Compound 1 (0.5 g, 1.43 mmol) dissolved in 1,4-dioxane:water (1:1, v/v, 10 mL) and dowex resin 50w (0.89 g) was added and resulting solution was stirred at 100° C. for 1 h. TLC was used to monitor reaction completion, and the dowex resin was filtered off on cotton and then concentrated under reduced pressure. The crude residue was purified by column chromatography (CH₂Cl₂/MeOH, 1:10) to afford complex mixtures of product (0.2 g, 52%) as a white solid, which was processed next step without any purification.

Synthesis of compound 3: Compound 2 (200 mg, 0.739 mmol) was dissolved in DMF (5 mL) and benzaldehyde dimethyl acetal (144.4 μL, 0.961 mmol) and p-Toluenesulfonic acid (25.5 mg, 0.147 mmol) were added at RT. The resulting mixture was stirred at 45° C. for 12 h. TLC was used to monitor reaction completion and the RM was diluted with ethyl acetate and washed with NaHCO₃ and brine solution. The combined organic layer was dried over Na₂SO₄, filtered, and concentrated under reduced pressure. The crude residue was purified by column chromatography (Ethyl acetate/Hexane, 1:4) to afford compound 3 (182 mg, 68%) as a white solid.

Synthesis of compound 4: Compound 3 (227 mg, 0.633 mmol) was dissolved in CH₂Cl₂ (5 mL) and DMAP (15.5 mg, 0.126 mmol), TEA (883 μL, 6.33 mmol) and Ac₂O (239.5 μL, 2.53 mmol) were added dropwise and the resulting mixture was stirred for 3 h. TLC was used to monitor reaction completion and the RM was diluted with ethyl acetate and washed with brine solution. The combined organic layer was dried over Na₂SO₄, filtered, and concentrated under reduced pressure. The crude residue was purified by column chromatography (Ethyl acetate/Hexane, 1:6) to afford compound 4 (251 mg, 89%) as a white solid.

Synthesis of compound 5: Compound 4 (200 mg, 0.452 mmol) dissolved in CH₂Cl₂ (4 mL) was cooled to 0° C., and aqueous trifluoroacetic acid (11:25, v/v, 2 mL) was added dropwise. The reaction mixture was stirred at room temperature for 4 h. TLC was used to monitor reaction completion, then neutralized with ice-cooled saturated aqueous NaHCO₃. After dilution with CH₂Cl₂, the organic phase was washed with water. The combined organic layer was dried over Na₂SO₄, filtered, and concentrated under reduced pressure. The crude residue was purified by column chromatography (Ethyl acetate/Hexane, 1:4) to afford compound 5 (102 mg, 63%) as a colorless oil.

Synthesis of compound 6: Compound 5 (102 mg, 0.287 mmol) was dissolved in CH₂Cl₂ (3 mL) and DMAP (7.03 mg, 0.057 mmol), TEA (401.2 μL, 2.88 mmol) and Ac₂O (109 μL, 1.15 mmol) were added dropwise and the resulting mixture was stirred for 3 h. TLC was used to monitor reaction completion and the RM was diluted with ethyl acetate and washed with brine solution. The combined organic layer was dried over Na₂SO₄, filtered, and concentrated under reduced pressure. The crude residue was purified by column chromatography (Ethyl acetate/Hexane, 1:6) to afford compound 6 (106 mg, 84%) as a white solid.

Synthesis of compound 7: Compound 6 (106 mg, 0.241 mmol) was dissolved in EtOAc and Pd/C (40 mg) was added. The resulting mixture was stirred for 12 h under an H₂ atmosphere. TLC was used to monitor reaction completion, and the Pd/C was filtered on a celite bed washed with EtOAc (5×). The crude product was concentrated under reduced pressure and purified by column chromatography (Ethyl acetate/Hexane, 1:5) to afford compound 7 (42 mg, 50%) as a white solid β-isomer.

Synthesis of compound 8: Compound 7 (20 mg, 0.072 mmol) was dissolved in CH₂Cl₂ (2 mL) and cooled to −17° C. Pyridine (48 μL, 0.602 mmol) and Tf₂O (12.2 μL, 0.072 mmol) was added dropwise. The resulting mixture was stirred for 1 h. TLC was used to monitor reaction completion and the RM was diluted with EtOAc and washed with 1N HCl solution (4×). The combined organic layer was dried over Na₂SO₄, filtered, and concentrated under reduced pressure. The crude residue was purified by column chromatography (Ethyl acetate/Hexane, 1:6) to afford compound 8 as a colorless oil.

### Preparation 8

### Synthesis of 3FDGal from 3-Deoxy-3-fluoro-1,2:5,6-bis-O-(1-methylethylidene)-α-D-galactofuranose in QMA cartridge

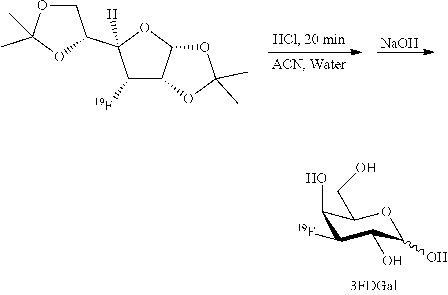

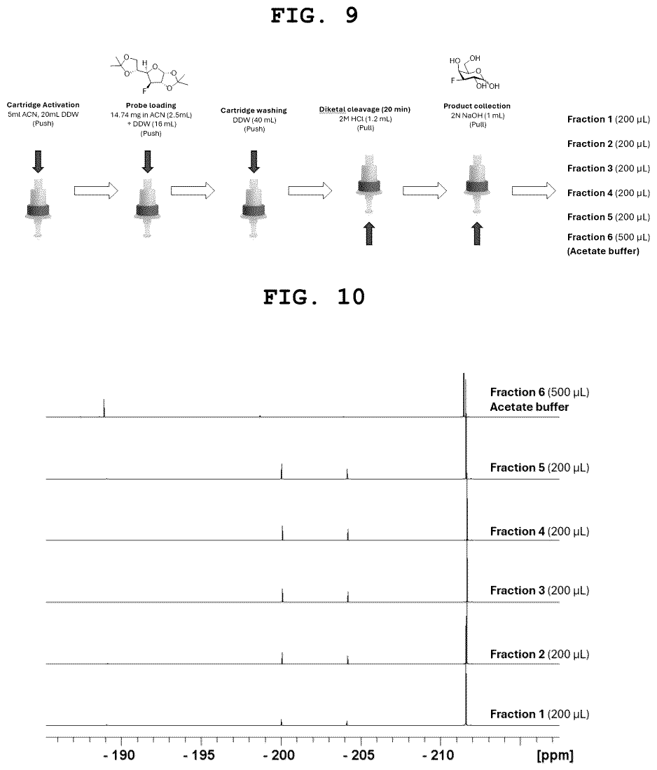

The flowchart of the synthetic process of preparation of 3FDGal from 3-Deoxy-3-fluoro-1,2:5,6-bis-O-(1-methylethylidene)-α-D-galactofuranose in a Quaternary Methyl Ammonium (QMA) cartridge is depicted in FIG. 9. The protected diketal fluorosugar intermediate was obtained after substituting the triflate protecting group (compound of Preparation 6) with a fluoride from a CsF salt.

The diketal fluorosugar intermediate (3-Deoxy-3-fluoro-1,2:5,6-bis-O-(1-methylethylidene)-α-D-galactofuranose) (14.74 mg) was dissolved in acetonitrile (2.5 mL) and water (16 mL). An aliquot of the obtained solution (0.25 mL) was withdrawn for ¹⁹F-NMR analysis.

The QMA cartridge (Sep-Pak Accell Plus QMA 6 cc Vac Cartridge) was activated by sequentially flushing with acetonitrile (5 mL) and water (20 mL). The diketal solution was then passed through the preconditioned QMA cartridge under controlled flow conditions. At this stage, the fluorinated, protected sugar is expected to remain retained on the cartridge due to its hydrophobic protecting groups. The cartridge with the bound compound was rinsed with water (40 mL) to remove any unbound material. At this stage, only the diketal remains on the cartridge.

2N hydrochloric acid (1.2 mL) was added to the top of the QMA cartridge to initiate the ketal cleavage reaction. The contact between the HCl and the stationary phase was maintained for exactly 20 minutes at room temperature (RT).

A syringe was connected to the top outlet of the cartridge, and 2N sodium hydroxide (0.2 mL) was slowly drawn to ensure thorough elution and complete neutralization of any residual acidic species within the cartridge.

The first 1 mL of the eluted solution (five fractions of 0.2 mL each) contained the product 3-fluoro-3-deoxy-galactose (3FDGal), as determined by ¹⁹F-NMR (FIG. 10).

### Preparation 9

### Synthesis of 3FDXyl

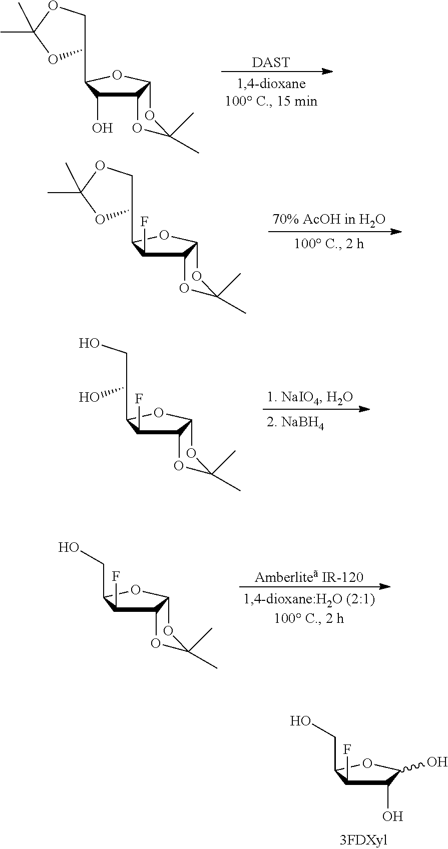

Preparation of 3-fluoro-3-deoxy-D-xylose was performed from ketal-protected 1, 2:5,6-Di-O-isopropylidene-α-D-allofuranose. The diketal (3 g, 11.53 mmol) was dissolved in Dioxane (30 mL) and heated to 100° C. The resulting solution was treated with Diethylaminosulfur trifluoride (DAST) (4.57 mL, 34.58 mmol) at 100° C. for 15 minutes. The ketal protecting groups were sequentially removed from the resulting 3-fluoro-3-deoxy-sugar diketal using treatment with acetic acid (70% AcOH in H₂O, 25 mL) at 100° C. for 2 hours, followed by sodium metaperiodate 508 mg, 2.38 mmol) and sodium borohydride (67.3 mg, 1.78 mmol). Further purification on cation exchange resin Amberlite™ IR-120 with Dioxane/Water—2/1 at 100° C. for 2 hours gave 3-fluoro-3-deoxy-D-xylose with 48% yield and 98% purity. The obtained 3-fluoro-3-deoxy-D-xylose was then crystallized from ethanol in diethyl ether solution.

### Preparation 10

### Synthesis of 3FDRib

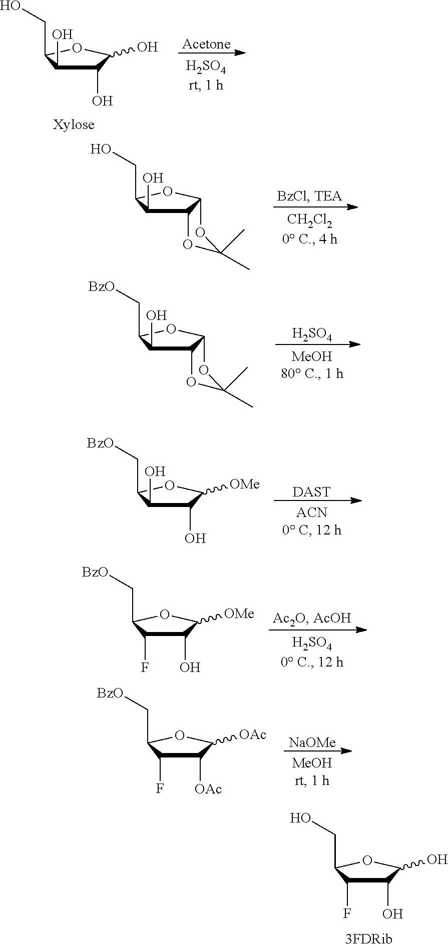

Xylose (2.2 g, 14.65 mmol) was treated with sulfuric acid (2.19 mL, 41.03 mmol) in Acetone (40 mL) at RT for 1 h. Treatment of the ketal-protected sugar with benzoyl chloride (0.34 mL, 2.95 mmol) triethyl amine (0.4 mL, 3.18 mmol) in DCM (15 mL) at 0° C. for 4 hours gave the ketal-benzoyl protected sugar. Ketal deprotection by sulfuric acid (69 μL, 1.3 mmol) in methanol (10 mL) at 80° C. for 1 hour gave the possibility to fluorinate the obtained methyl substituted sugar with Diethylaminosulfur trifluoride (DAST) (0.66 mL, 5.02 mmol) in acetonitrile (14 mL) at 0° C. for 12 hours. Further acetylation of the 3-fluoro-3-deoxy-methyl-benzoyl ribose with acetic anhydride (1.7 mL, 17.48 mmol) in acetic acid (0.2 mL, 3.89 mmol) and sulfuric acid (124 μL) at 0° C. for 12 hours followed by hydrolysis with sodium methoxide (85 mg, 1.59 mmol) in methanol (8 mL) at RT for 1 h to remove acetyl and benzoyl protecting groups, afforded 3-fluoro-3-deoxy-ribose with 2.6% overall yield and 98% purity.

### Example 1

### 3FDG, 3FDS and 3FDGA are Differentiable by ¹⁹F-NMR Spectroscopy and are Imageable by ¹⁹F-MRI

A series of phantom experiments were performed. 10 mM aqueous solutions of 3FDG, 3FDS and 3FDGA were separately added to three tubes. An aqueous solution consisting of a mixture of the three compounds (each at a concentration of 10 mM) was added to a fourth tube.

¹⁹F{¹H} NMR measurements were performed as described above. The characteristic ¹⁹F-NMR chemical shifts are depicted at the top of FIG. 2A, indicating ¹⁹F-NMR shifts assigned to 3FDGA and 3FDS at −209 and −213 ppm. The chemical shift assigned to 3FDG is at −195 ppm.

The spectral data recorded, which indicates the different ¹⁹F-NMR chemical shifts of the three fluorinated compounds, forms the basis for spatially mapping the location of the compounds dissolved in the four tubes with a single ¹⁹F-MRI mCSSI-based FMI acquisition that was described above. The left-hand image was created by ¹H-MRI (the tubes are indicated by white circles) whereas the right-hand image shows the FMI data overlaid on the ¹H-MRI data, with distinct colors assigned to the three compounds as described above. Note also the imageability of the mixed sample.

### Example 2

### 3FDGal, 3FDGalA and 3FDGtol are Differentiable by ¹⁹F-NMR Spectroscopy and are Imageable by ¹⁹F-MRI

A series of phantom experiments were performed. 10 mM aqueous solutions of 3FDGal, 3FDGalA and 3FDGtol were separately added to three tubes. An aqueous solution consisting of a mixture of the three compounds (each at a concentration of 10 mM) was added to a fourth tube.

¹⁹F{¹H} NMR measurements were performed as described above. The characteristic ¹⁹F-NMR chemical shifts are depicted at the top of FIG. 2B, indicating ¹⁹F-NMR shifts assigned to 3FDGalA and 3FDGtol at −205 and −211 ppm. The chemical shift assigned to 3FDGal is at −199 ppm.

The spectral data recorded, which indicates the different ¹⁹F-NMR chemical shifts of the three fluorinated compounds, forms the basis for spatially mapping the location of the compounds dissolved in the four tubes with a single ¹⁹F-MRI mCSSI-based FMI acquisition that was described above. The left-hand image in FIG. 2B was created by ¹H-MRI (the tubes are indicated by white circles) whereas the right-hand image shows the FMI data overlaid on the 1H-MRI data, with distinct colors assigned to the three compounds as described above. Note also the imageability of the mixed sample.

### Example 3

### In Vivo Study of 3FDGal and 3FDG Metabolism

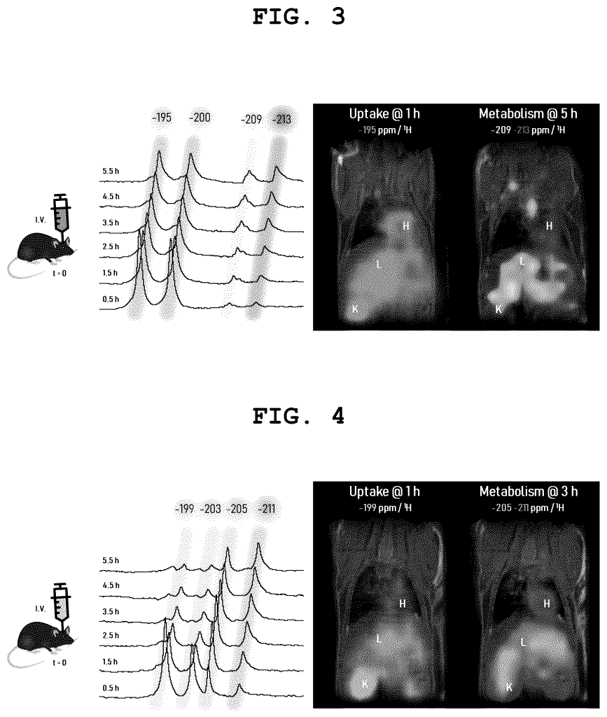

The in vivo metabolism of systemically administered 3FDG and 3FDGal was studied. To this end, mice were injected with 3FDG (n=4, results of one representative mouse are shown in FIG. 3) or 3FDGal (n=4, results of one representative mouse shown in FIG. 4).

### Experimental Protocol

Mice were kept in a daily controlled room at the Weizmann Institute of Sciences (WIS) animal facility with a surrounding relative humidity level of 50±10% and a temperature of 22±1° C., with a 12/12 cycle of dark and light phases.

Eight healthy C57BL/6 male mice underwent in vivo imaging under anesthesia as described above. In all cases, at t=0, either 3FDG or 3FDGal was administered intravenously as the molecular probe (15 mg in 70 μl of PBS, ~0.6 g/kg). FMI was performed repeatedly starting at t=30 min post-injection, applying multi-chemical shift selective imaging (mCSSI), a RARE sequence variant that allows recording multiple metabolic images concurrently as described above. Before the initial FMI step, in between the FMI steps, and after the last FMI step, anatomical 1H-MRI and non-localizing ¹⁹F-NMR spectra were recorded.

### Results

The images are shown in FIGS. 3 and 4 for the 3FDG-injected mice and 3FDGal-injected mice, respectively. In all mice studied, the injection of the 3FD-sugar revealed 3FD-acid and 3FD-polyol generation in vivo. Based on the obtained ¹⁹F-NMR spectral data, 3FDGal metabolism resulted in two prominent, well-defined peaks corresponding to the chemical shifts of 3FDGalA and 3FDGtol, which were localized on 2D-FMI (voxel size, 2×2×24 mm³) mainly in the liver and heart, respectively (FIG. 4). But for 3FDG injected mice, while a clear 3FDS peak was observed, the 3FDGA peak was “contaminated” with that of additional ¹⁹F-carrying metabolites, suggesting that 3FDGA undergoes further metabolism in vivo.

The results indicate that the two 3-fluoro-3-deoxy-sugars tested—3FDGal and 3FDG—are both suitable for use in ¹⁹F-based metabolic imaging. But 3FDGal, owing to its higher and preferable metabolic conversion to the corresponding polyol and acid, is a more efficient imaging agent compared to 3FDG.

The efficient metabolic conversion of 3FDGal to 3FDGalA in the liver and to 3FDGtol in the heart was further confirmed with the aid of localized ¹⁹F-MR spectroscopy, as shown in FIG. 5. ¹H-MRI images, ¹⁹F-images, and ¹⁹F-images overlaid on the anatomic background provided by 1H-MRI are shown for the liver (where 3FDGalA accumulated, the upper set of images) and the heart (where 3FDGtol accumulated, the lower set of images).

Additionally, the in vivo high metabolite yield of 3FDGal served for acquiring higher resolution 3D-FMI data (n=4), recording metabolites maps with voxel size of 2×2×3 mm³, concurrently reporting the spatial distribution of sugar oxidizing and reducing enzymes in vivo and in real-time, as indicated by the images shown in FIG. 6.

### Example 4

### In Vitro Study of 3FDGal Metabolism in Different Cancer Cell Lines

For in vitro incubation studies, seven different cell lines (6×10⁶ cells) were incubated with 5 mM 3FDGal for 4 hours (n=3 each, one representative repetition is presented per cell line in the appended drawings). The cell lines studied were MC38, N2A, MDA-MB-231, HEK293, HepG2, A549 and LNCap.

For cell incubation with a ¹⁹F-labeled probe, 6×10⁶ cells were added to a 12 mL FACS tube containing 1.5 mL of medium [DMEM, 10% FBS, 1% L-Glutamine, 1% Penicillin/Streptomycin] and 5 mM of the ¹⁹F-labeled probe, i.e., 3FDGal. Incubation lasted 4 h, with gentle resuspension at 1, 2, and 3 h. At 4 h, after centrifugation (7 min, 200 G, 25° C.), 540 μL from the incubating supernatant were added to 60 μL of D₂O [labeled as the extracellular (EC) content]. The remaining supernatant was discarded, and the pellet was washed twice with PBS by resuspending it in a 7 mL PBS, followed by centrifugation for 7 min at 200 G, 25° C., and discarding the supernatant. To lyse the cells, 610 μL of lysis RIPA buffer+1:1000 Protease Inhibitor was added to the pellet. The tubes were shaken gently on ice for 20 min (with gentle vortexing at 5, 10, and 15 min). After centrifugation of the resultant content (20 min, 10,000 G, 4° C.), 540 μL from the lysate was added to 60 μL of D²O [labeled as the intracellular (IC) content].

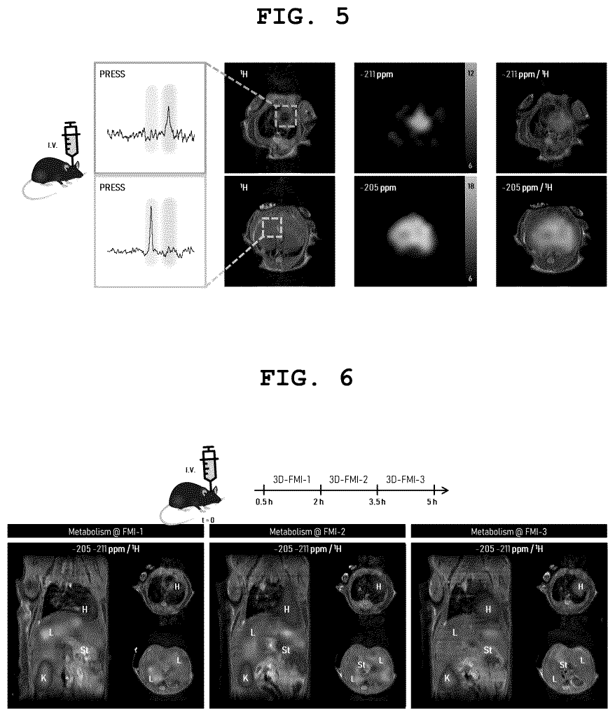

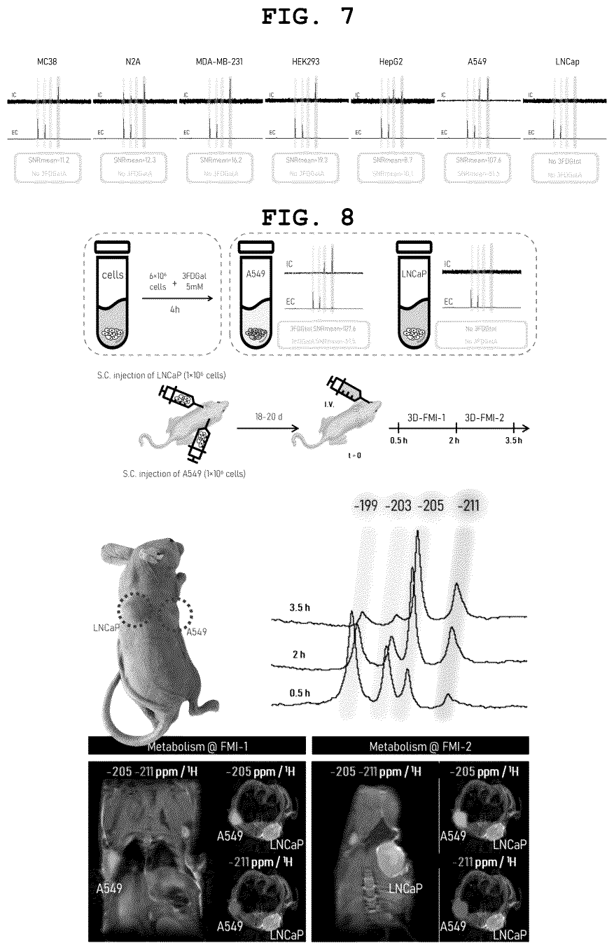

The extracellular (EC) medium and the intracellular (IC) content were studied by ¹⁹F-NMR. The results shown in FIG. 7 reveal prominent cell-specific differences in the SNR values calculated for the ¹⁹F-NMR peak corresponding to 3FDGalA and 3FDGtol (−205 and −211 ppm, respectively, labeled gold and magenta in FIG. 7). Of the seven cell lines studied, 3FDGalA was detected only in HepG2 and A549 cells, while varying concentrations of 3FDGtol were generated by all cells except for LNCaP (the only cell showing no in vitro 3FDGal metabolism). Thus, ¹⁹F-NMR enables cell categorization based on the metabolites 3FDGalA and 3FDGtol IC SNRs measured by the protocol described above, as shown in FIG. 7.

### Example 5

### In Vivo Study of 3FDGal Metabolism in Tumor-Bearing Mice

An in vivo study was performed to determine whether the different metabolic fingerprint shown by A549 and LNCaP cells upon transport of 3FDGal into the cells in vitro, is observed in vivo. To this end, three athymic nude mice were subcutaneously injected with A549 and LNCaP cells in the right and left posterior aspects of their chest, respectively, to induce proliferating subcutaneous tumors.

### Experimental Protocol

Mice were kept in a daily controlled room at the Weizmann Institute of Sciences (WIS) animal facility with a surrounding relative humidity level of 50±10% and a temperature of 22±1° C., with a 12/12 cycle of dark and light phases. To induce proliferating subcutaneous A549 and LNCaP tumors, athymic nude mice were subcutaneously injected 1×10⁶ A549 cells (in 150 μl PBS) and 1×10⁶ LNCaP cells in the right and left posterior aspects of their chest, respectively. The subcutaneous injections were performed under anesthesia (isoflurane). The mice were then monitored daily until visible tumors could be seen and assessed with the in vivo MRI setting. The three nude athymic tumor-bearing mice underwent in vivo imaging under anesthesia as described above. In all cases, at t=0, 3FDGal was administered intravenously as the molecular probe (15 mg in 70 μl PBS, ~0.6 g/kg). 3D-FMI was performed repeatedly starting at t=30 min post-injection, applying multi-chemical shift selective imaging (mCSSI), as described above. Before the initial FMI step, in between the FMI steps, and after the last FMI step, anatomical 1H-MRI and non-localizing ¹⁹F-NMR spectra were recorded. The experimental protocol, consisting of subcutaneous injections of cells, intravenous administration of 3FDGal and imaging are shown in FIG. 8 (top section).

### Results

The images acquired through the in vivo 3D-FMI are shown in the bottom section of FIG. 8. The images revealed the generation of both 3FDGalA and 3FDGtol (gold and magenta ¹⁹F-MRI signals, respectively, overlaid on anatomical ¹H-MRI) in the A549 tumor and no 3FDGal metabolism in the LNCaP tumor (grey area), establishing the ability to distinguish between tumors on the molecular level applying FMI with 3FDGal. That is, 3FDGal can selectively and efficiently report the spatial distribution of sugar oxidizing and reducing enzymes in different cancer types, in vivo and in real time. This way, categorization of tumors becomes available based on their molecular composition, regardless of their dependence on glycolysis, simultaneously targeting two different metabolic pathways, and visualizing them in a multiplexed manner.

### Example 6

### Identifying the Human Enzymes Responsible for 3FDG and 3FDGal Cellular Metabolism

To identify the human enzymes responsible for the redox transformations of 3FDG and 3FDGal to their metabolic fates, gain-of-function studies in HEK-293 cells overexpressing candidate redox biomarkers were conducted, using an established transfection/incubation protocol which was previously reported by Allouche-Arnon, H. et al., Nat Biotechnol 40, 1143-1149, 2022 and Allouche-Arnon, H. et al., Journal of the American Chemical Society 146, 31624-31632 (2024), both of which are incorporated herein by reference.

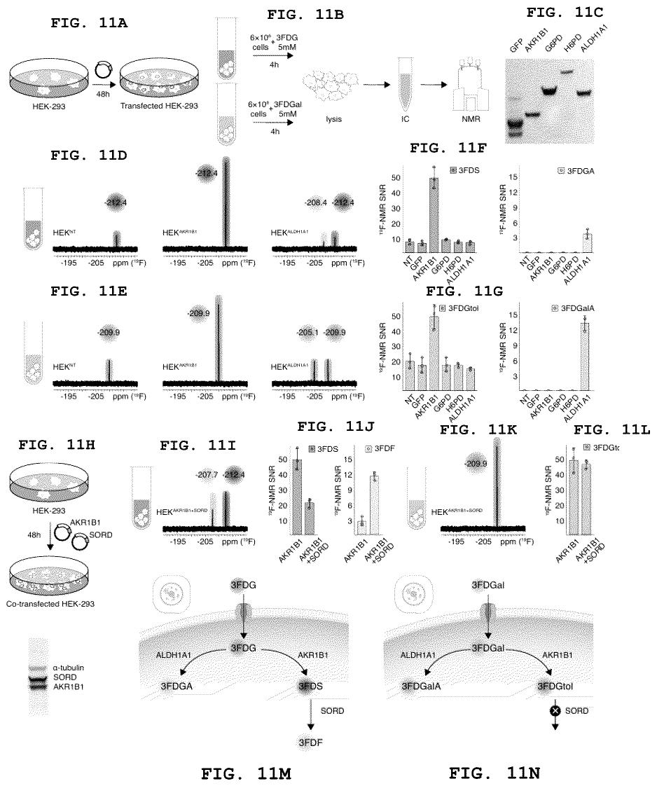

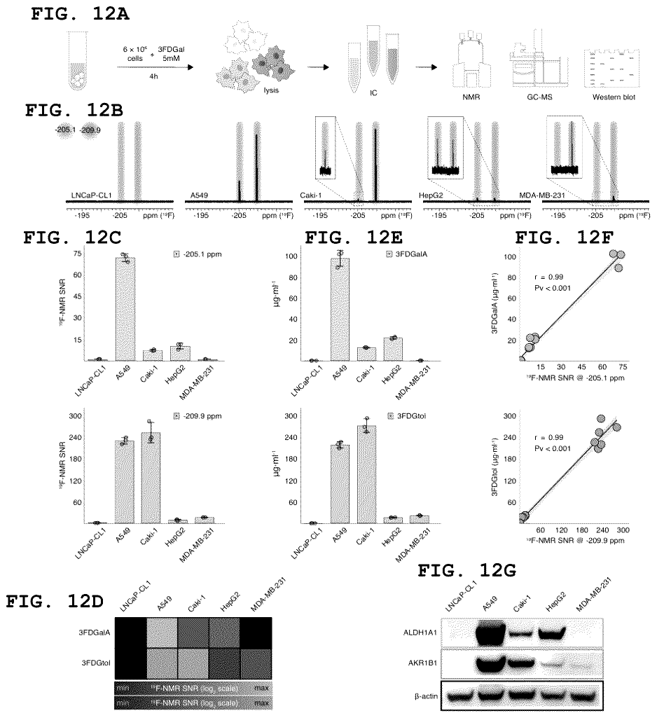

After transfection with plasmids that encode for the expression of the candidate enzymes (as schematically shown in FIG. 11A), cells were incubated with either 3FDG or 3FDGal (6×10⁶ cells, 5 mM sugar, for four hours), lysed, and analyzed using ¹⁹F-NMR to determine their intracellular (IC) ¹⁹F-content (FIG. 11B). Western blot analysis confirmed the successful expression of the examined proteins (FIG. 11C). Non-transfected cells (NT) were used to establish the baseline metabolic activity, and cells transfected with green fluorescent protein (GFP) served to examine the effect of the transfection process itself on experimental observations.

Following the four hours incubation period of the control cells with either 3FDG or 3FDGal (IC₅₀>10 mM), the ¹⁹F-NMR spectra of the IC content indicated a minute level of 3FDS (FIG. 11D, red, −212.4 ppm) or 3FDGtol (FIG. 11E, magenta, −209.9 ppm), respectively, with no significant difference between NT cells and those expressing GFP. No peaks characterizing 3FDGA or 3FDGalA were evident on the IC spectra, indicating negligible baseline oxidation of the probes in HEK-293 cells. Moreover, the lack of 3FDG or 3FDGal IC peaks implied that the 3-fluorosugars are not retained in the cells if not metabolized. Notably, overexpression of AKR1B1, the major human aldose reductase, significantly increased the production of 3FDS from 3FDG (FIG. 11F; SNR of 48.7±7 in HEK-293^{AKR1B1} compared to 7.3±1.7 in HEK-293NT cells, p-value <0.001) and 3FDGtol from 3FDGal (FIG. 11G; SNR of 48.4±8 in HEK-293^{AKR1B}1 compared to 19.3±4.9 in HEK-293NT cells, p-value=0.006), confirming its role in the reduction of the 3-fluorosugars and consistent with previous reports.

To identify human enzymes that can intracellularly oxidize 3FDG and 3FDGal to their corresponding acids, the activity of 3FDG and 3FDGal with two human enzymes known to exhibit glucose-1-dehydrogenase activity was first examined. The one, glucose-6-phosphate dehydrogenase (G6PD), which was previously speculated to be the enzyme that oxidizes 3FDG to 3FDGA showed no activity with either of the examined 3-fluorosugars (FIG. 11F, FIG. 11G). Upon incubation of HEK-293^{G6PD} cells with either 3FDG or 3FDGal, the ¹⁹F-NMR of the IC content of these cells showed no indication of the formation of either 3FDGA (δ=−208.4 ppm) or 3FDGalA (δ=−205.1 ppm), respectively.

Overexpressing hexose-6-phosphate dehydrogenase/glucose 1-dehydrogenase (H6PD), which is known to have a broader substrate specificity compared to G6PD also showed no conversion of the intracellular 3FDG or 3FDGal to the respective acids (FIG. 11F, FIG. 11G).

Finding that G6PD and H6PD do not metabolize the 3-fluorosugars, it was hypothesized that, similar to AKR1B1, the oxidative transformation might require an enzyme that targets sugar aldehydes. Testing this, ALDH1A1, also known to be able to oxidize 3-deoxyglucosone in HEK-293 cells was overexpressed. In both 3FDG- and 3FDGal-treated HEK-293^{ALDH1A1} cells, ¹⁹F-NMR peaks of the corresponding acids (3FDGA, δ=−208.4 ppm, marked in yellow in FIG. 11D, and 3FDGalA, δ=−205.1 ppm, marked in gold in FIG. 11E) confirmed the oxidative role of ALDH1A1 in the probes' metabolism. Quantifying these results, it was found that 3FDGalA is accumulated at much higher levels in 3FDGal-treated HEK-293^{ALDH1A1} cells compared to 3FDGA accumulated in 3FDG-treated HEK-293ALDH1A1 cells (~3.6 fold more, p-value <0.001, FIG. 11F, FIG. 11G).

Since FMI studies rely on cellular accumulation of high levels of the obtained redox metabolites and the ability to spectrally resolve their ¹⁹F-NMR signals, the hypothesis that intracellular 3FDS can be further metabolized to 3-fluoro-3-deoxy-fructose (3FDF) by sorbitol dehydrogenase (SORD,) was also examined. Co-overexpression of both AKR1B1 and SORD (FIG. 11H) revealed that cellular levels of 3FDS in 3FDG-treated HEK-293^{AKR1B1+SORD} cells were significantly lower than the levels recorded in 3FDG-treated HEK-293^{AKR1B1} cells (FIG. 11I, FIG. 11J, SNR at δ=−212.4 ppm of 20.5±3 versus 48.7±7, p-value=0.003). Not only that, ¹⁹F-NMR spectra indicated intracellular production of 3FDF (FIG. 11I, FIG. 11J, SNR at δ=−207.7 ppm27 of 11.4±0.8), which resonates only 0.7 ppm downfield from the peak of 3FDGA (δ=−208.4 ppm), a chemical shift difference that is most likely not applicable for multiplex in vivo ¹⁹F-MRI studies.

In contrast to the 3FDS-to-3FDF conversion, SORD showed no oxidation of AKR1B1-produced 3FDGtol, with cellular levels of 3FDGtol in 3FDGal-treated HEK-293^{AKR1B1+SORD} and HEK-293^{AKR1B1} cells being similar (FIG. 11K, FIG. 11L). This leaves the intracellular levels of 3FDGtol high even when AKR1B1 is co-expressed with SORD. The fact that galactitol dehydrogenase, the enzyme that catalyzes the oxidation of unlabeled galactitol (and might oxidize 3FDGtol), has no known human homolog, makes this conversion less likely in mammalian systems, and positions 3FDGal as a superior probe over 3FDG for accurate mapping of the expression of the reducing enzyme AKR1B1.

Overall, the suggested trapping cellular mechanisms of 3FDG and 3FDGal are sketched in FIG. 11M-FIG. 11N, positioning 3FDGal as a robust and reliable probe for the simultaneous detection of AKR1B1 and ALDH1A1 activities. The redox products of 3FDGal were found to be intracellularly stable (not further metabolized), spectrally distinct from one another (Δω~5 ppm), and directly attributable to ALDH1A1 (via 3FDGalA) and AKR1B1 (via 3FDGtol) activities, leading to the selection of 3FDGal as the exclusive probe for subsequent FMI studies exploring cancer metabolism.

### Example 7

### 3FDGal as a Molecular Probe for Cancer ¹⁹F-NMR Fingerprinting

Further to Example 4, the goal of the study reported in the present example was to evaluate if the ¹⁹F-NMR characteristics of 3FDGal metabolism could be used to molecularly typify cancers through distinct redox metabolic fingerprints.

To this end, five well-characterized human cancer cell lines were tested: A549 (lung); MDA-MB-231 (breast); LNCaP-CL1 (prostate); HepG2 (liver); and Caki-1 (kidney), partially overlapping with the cell lines used above. In this case as well, cells were incubated with 3FDGal, this time followed by multimodal analysis of their IC content using ¹⁹F-NMR, GC-MS, and Western blotting (schematically shown in FIG. 12A).

Reviewing the ¹⁹F-NMR spectra of the IC of the incubated cells (FIG. 12B), each cell line exhibited a unique metabolic fingerprint, reflected in characteristic SNR values calculated for the peaks of 3FDGalA at δ=−205.1 ppm and of 3FDGtol at δ=−209.9 ppm in the ¹⁹F-NMR spectra (FIG. 12C).

Summarized as a dual-color metabolic heatmap, one can clearly see the different fingerprint obtained for each cell line examined (FIG. 12D). For example, the A549 cells showed very strong ¹⁹F-NMR signals of both 3FDGalA (SNR of 71.4±2.8 vs. SNR of 10.1±2.1 obtained for HepG2 cells, p-value <0.001) and 3FDGtol (SNR=228.7±9.6 vs. SNR=16.2±0.2 obtained for MDA-MB-231 cells, p-value <0.001). In contrast, incubating Caki-1 cells with 3FDGal resulted in a very strong ¹⁹F-NMR peak of the reduced form 3FDGtol (SNR=250.8±28.3 vs. SNR=8.7±2.3 obtained for HepG2 cells, p-value <0.001) with a noticeably lower level of the oxidized metabolite 3FDGalA (SNR=7.1±0.6).

Strikingly, the prostate cancer cell line examined, LNCaP-CL1, showed no indication of the production of either 3FDGalA or 3FDGtol in the ¹⁹F-NMR spectrum of its IC content (FIG. 12D).

To validate that the ¹⁹F-NMR fingerprints obtained precisely represent the amounts of 3FDGalA and 3FDGtol produced in the cells, the same samples were analyzed with gas chromatography-mass spectrometry (GC-MS). Synthetic 3FDGal, 3FDGalA, and 3FDGtol were used to optimize and calibrate GC-MS measurements. The IC concentrations of 3FDGalA and 3FDGtol determined per cell type (FIG. 12E) showed patterns similar to those obtained from the ¹⁹F-NMR measurements (FIG. 12C). This similarity was quantified by plotting the GC-MS extracted data against those obtained from ¹⁹F-NMR, revealing a strong correlation in both cases (Pearson's r=0.99, p-value <0.001, FIG. 12F), and reflecting the validity of ¹⁹F-NMR fingerprinting to classify different cancers based on 3FDGalA or 3FDGtol IC content. Western blot analysis of all examined cell lines (FIG. 12G) showed that 3FDGalA levels aligned with ALDH1A1 expression levels (A549>>HepG2>Caki-1>>MDA-MB-231~LNCaP-CL1), and 3FDGtol levels aligned with AKR1B1 expression levels (A549~Caki-1>>MDA-MB-231~HepG2>>LNCaP-CL1). Overall, the experimental datasets summarized in FIG. 12 confirm that the redox ¹⁹F-NMR fingerprints of introduced 3FDGal accurately reflect each cell line's enzymatic phenotype, and that 3FDGal has the potential to trace two different redox biomarkers in cancer, ALDH1A1 and AKR1B1.

### Example 8

### In Vivo FMI for Cancer Fingerprinting

Establishing the in vivo FMI platform that employs ¹⁹F-MRI to map 3FDGal and its metabolites (3FDGalA and 3FDGtol), it was next examined whether the “dual-color” ¹⁹F-NMR fingerprints of cancer cells introduced with 3FDGal (as shown herein above) could be detected in vivo to report on dual-biomarker expression (ALDH1A1 and AKR1B1) and noninvasively classify cancers. To this end, experiments similar to those detailed in Example 5 (above) were performed, while expanding the analyses thereof.

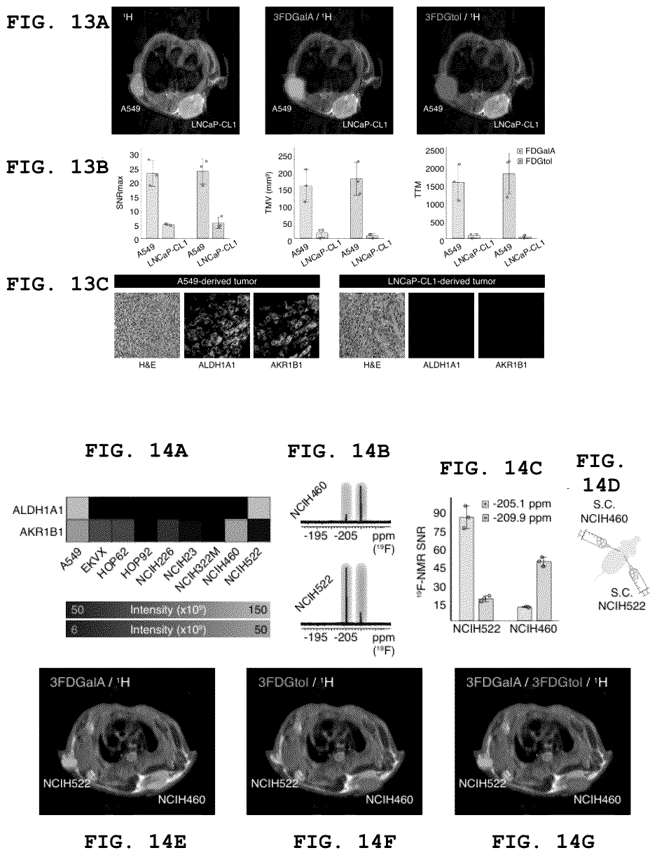

Briefly, bilateral tumor xenografts in three nude athymic mice were established, using A549 and LNCaP-CL1 cells (as schematically shown in FIG. 8). These two lines were shown to have contrasting redox biomarker profiles: ALDH1A1^{high}/AKR1B1^{high} for A549, and ALDH1A1^{low}/AKR1B1^{low} for LNCaP-CL1 (as shown herein). During the third week after subcutaneous inoculation of the cells, the examined mice underwent two consecutive 3D-FMI scans following 3FDGal injection (as schematically shown in FIG. 8). The overall imaging session length was set to 3.5 hours post-injection (p.i.) as the IC₅₀ of 3FDGal at 4 hours was found to be >10 mM in both cell lines. In all mice, anatomical 1H-MR images clearly located the tumors (FIG. 13A), with no ¹H-MRI-based data suggesting any molecular or metabolic difference between them. In contrast, ¹⁹F-MRI-based 3D-FMI maps revealed a clear difference between the two tumors. While A549 tumors actively transform the systematically administered 3FDGal into both 3FDGalA (gold signal at the 3D-FMI maps) and 3FDGtol (magenta signal at the 3D-FMI maps), LNCaP-CL1 tumors showed no detectable metabolism of the probe.

For standardized image quantification, three image-based quantitative parameters were set to be recorded in the tumor VOIs: maximal SNR (SNRmax); total metabolite volume (TMV); and total tissue metabolite (TTM). While SNRmax is easy to record but lacks volumetric insights, TMV better reflects signal volume (above a defined SNR cutoff), and TTM sums up the SNR values in all voxels within the TMV, combining signal intensity and extent. Comparing these parameters between the two tumors showed significantly higher values in the A549 tumor (FIG. 13B). In the A549 tumor, analysis of both 3FDGalA and 3FDGtol maps indicated an increase in SNRmax, TMV, and TTM values when comparing 3D-FMI-1 data (acquired ~1 h p.i.) with 3D-FMI-2 data (obtained ~2.5 hours p.i.). Such increased 3FDGalA and 3FDGtol signals at the A549 tumor over time imply continuous conversion of the introduced probe and parenchymal accumulation of its metabolites. This quantitative analysis further confirmed FMI fingerprinting capabilities, illustrating the platform's multilayered ability to distinguish between tumors in terms of metabolite yield, intensities, and real-time kinetics. Finally, while similar to ¹H-MRI, H&E stain could report no specific molecular distinction between the excised tumors, immunostains for ALDH1A1 and AKR1B1 aligned with the in vivo FMI observations and confirmed the different molecular phenotypes: A549 tumors stained positive for both enzymes, whereas LNCaP-CL1 tumors were negative (FIG. 13C).

Overall, the experiments presented above demonstrate the successful application of FMI for cancer imaging in the complex multivariable in vivo setting. Without wishing to be bound by theory, the data indicate that ALDH1A1 and AKR1B1 expression levels are the principal determinants of 3FDGal-FMI cancer signatures.

Notably, the A549 cancer model exhibited strong 3FDGal-avidity despite its reported low [¹⁸F]2FDG-avidity highlighting the unique capacity of FMI to detect and trace pathologies that remain invisible to conventional molecular imaging methods.

### Example 9

### Using FMI to Reveal Dual-Enzymatic Profiles within Cancers of the Same Type

It was next aimed at examining the ability of FMI to reveal diverse dual-enzymatic profiles even within cancers of the same type. To this end, a proteomic database (Frejno, M. et al., Nature Communications 11, 3639 (2020), incorporated herein by reference) was used to generate a heatmap of the expression of AKR1B1 and ALDH1A1 across all non-small cell lung cancer (NSCLC) cell lines included in the National Cancer Institute-60 (NCI-60) panel (FIG. 14A), confirming the ALDH1A1^{high}/AKR1B1^{high} phenotype for A549. It was then also identified that NCIH522 and NCIH460 cell lines have opposite phenotypes, i.e., ALDH1A1^{high}/AKR1B^{low} and ALDH1A1^{low}/AKR1B1^{high}, respectively. Practically, in vitro incubation of both cell types with 3FDGal, followed by ¹⁹F-NMR of their IC content, revealed the above fingerprints (FIG. 14B-FIG. 14C). As shown in FIG. 14A, for NCIH522 cell line, the ¹⁹F-NMR dataset showed a 3FDGalA^{high}/3FDGtol^{low} pattern. In contrast, for NCIH460, a 3FDGalA^{low}/3FDGtol^{high} observation was depicted.

To further analyze these phenotypes, these cells were then inoculated into a nude athymic mouse to establish bilateral subcutaneous xenografts (according to the protocols detailed above). Following the same imaging protocol as above, 3D-FMI data overlaid on anatomical 1H-MRI data revealed similar imaging phenotypes: a 3FDGalA^{high}/3FDGtol^{low} combination of signals in the NCIH522-derived tumor and a 3FDGalA^{low}/3FDGtol^{high} phenotype in the NCIH460-derived one (FIG. 14D-FIG. 14F). These in vivo multicolor combinations mirrored the proteomic-based dual biomarker phenotypes of the tumors' cells-of-origin, highlighting the power of 3FDGal-FMI to distinguish metabolic subtypes within a single cancer category (specifically demonstrated here for NSCLC).

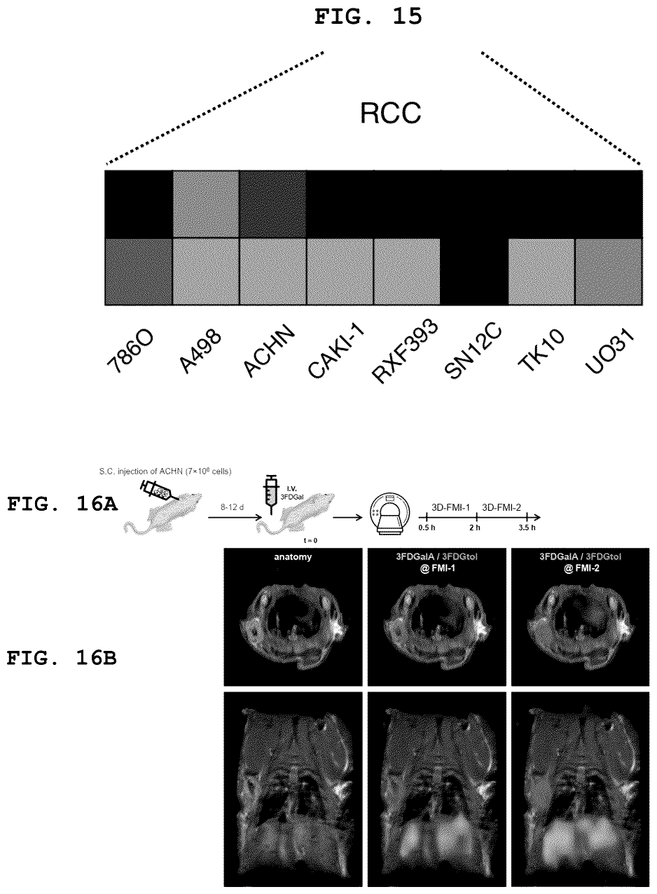

In addition, and taking the same experimental approach, the power of 3FDGal-FMI to distinguish metabolic subtypes within renal cell carcinoma (RCC) related cell lines was demonstrated, where a variable ALDH1A1 expression and almost uniform very high AKR1B1 expression were observed (FIG. 15). Furthermore, NMR and western blot data of 3FDGalA and 3FDGtol levels matched protein expression and the patterns were visualized noninvasively in xenografts (as further shown below).

### Example 10

### Non-Invasive Visualization of Enzymatic Profiles in an Animal Model of Renal Cell Carcinoma

FMI protocol was next developed for non-invasive imaging of tumors derived from ACHN cells relating to renal cell carcinoma (RCC).

Similar to the procedure reported above for tumor induction, sub cutaneous injection of ACHN cells (7×10⁶ cells) to mice was monitored as follows: 8-12 days from cells injection, 3FDGal was injected (i.v.) to mice followed by two FMI analyses, 3D-FMI-1 (conducted 0.5 hours post 3FDGal injection) and 3D-FMI-2 (conducted 2 hours post 3FDGal injection as schematically shown in FIG. 16A). The results are shown in FIG. 16B, where the areas characterized by presence of the 3FDGalA product are clearly distinguished from areas characterized by presence of the 3FDGtol product. Furthermore, the increase in the intensity of 3FDGalA and 3FDGtol through time is also clearly demonstrated.

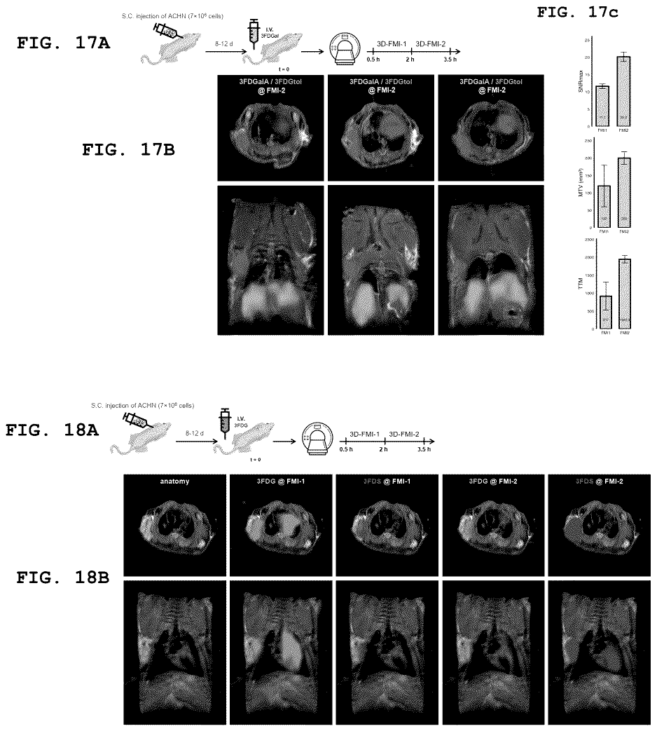

The above experiment was repeated in additional mice (by following the experimental procedure schematically shown in FIG. 17A), as shown in FIG. 17B where a clear detection of the xenografts is shown, visualizing the FMI phenotype of ACHN indicated above, namely, high AKR1B1 expression, mild ALDH1A1 expression which are based on both proteomic and western blot data; hence, generation of high 3FDGtol and mild 3FDGalA levels as shown in vitro and here in vivo. According to the quantification shown in FIG. 17C, the 3D-FMI-2 imaging provides a better imaging time point.

Next, imaging was performed on mice injected with 3FDG, using the above protocol (schematically shown in FIG. 18A). The results shown in FIG. 18B also demonstrate the ability to visualize uptake of 3FDG (green) followed by reduction to 3FDS (red) via high AKR1B1 activity.

Furthermore, a further FMI data acquisition was conducted at several timepoints, whereby a fast FMI acquisition time of only 20 minutes was validated, as detailed below.

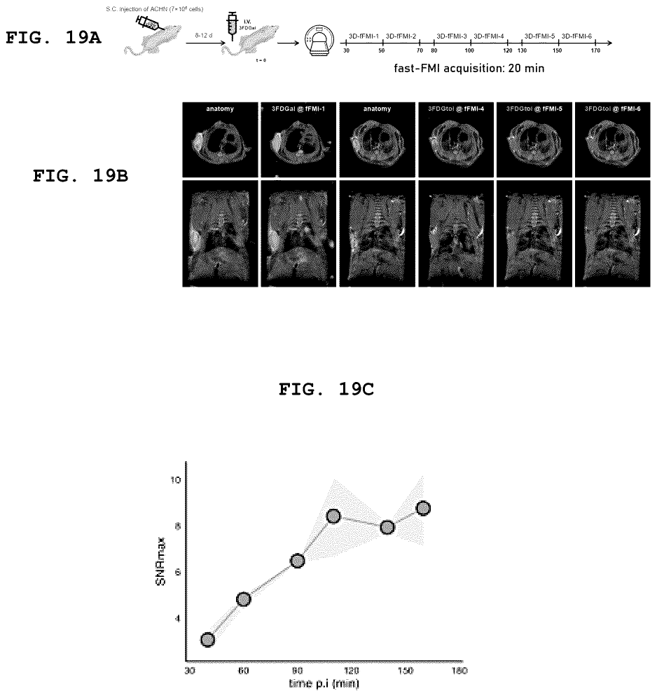

Imaging was performed on mice subcutaneously injected with ACHN (7×10⁶ cells) and 8-12 days later i.v. injected with 3FDGal (at t=0 as shown in the procedure scheme, FIG. 19A). Imaging acquisition time reduced to only 20 minutes per step (i.e., 20 minute imaging sessions) was performed at several time points post 3FDGal injection: at 30 (3D-fFMI-1), 50 (3D-fFMI-2), 80 (3D-fFMI-3), 100 (3D-fFMI-4), 130 (3D-fFMI-5) and 150 (3D-fFMI-6) minutes.

Following 3FDGal uptake, and the results shown in FIG. 19B, optimal results were observed between two and three (2-3) hours post injection, where substantial localization of 3FDGtol generation is shown in the tumors, despite the short acquisition time (FIG. 19C).

### Example 11

### Visualizing 3FDGal Metabolism in Glioma Cancer

Metabolism of 3FDGal with ¹⁹F-NMR was further analyzed in cancer cells derived from glioma (i.e., TBDRG-05MG), which is used to induce tumors in the brain.

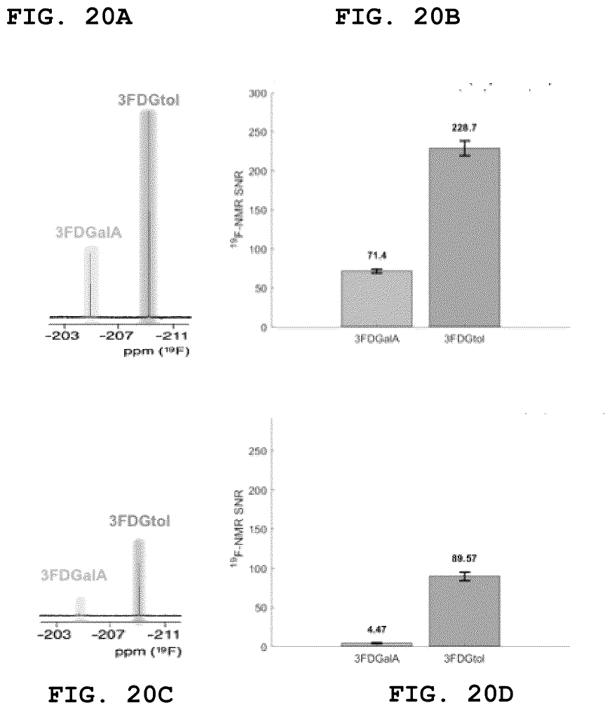

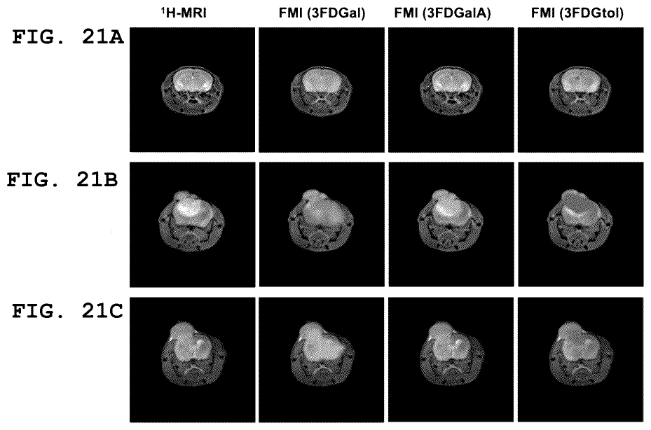

First, the metabolism derivatives of 3FDGal were analyzed in the cells, as shown in FIG. 20A-20D. Next, FIG. 21 shows a three-dimensional Fluorine Metabolic Imaging (3D-FMI) of the brain. Healthy mouse (top row, FIG. 21A) and mice inoculated with one of the indicated types of tumors in their brains (A549 is shown in FIG. 21B and TBDRG-05MG is shown in FIG. 21C) were studied using 3D-FMI 2 hours post-3FDGal injection. Shown are, from left to right, 4 different MRI data sets for each mouse representing an axial view of a single selected slice of the brain, anatomical ¹H-MRI, 3FDGal map (overlayed on the corresponding 1H-MRI data), 3FDGalA map (overlayed on the corresponding 1H-MRI data), and 3FDGtol map (overlayed on the corresponding 1H-MRI data). After intraperitoneal injection of 3FDGal (0.6 g/kg) at t=0, axial mCSSI-based 3D-FMI was acquired to simultaneously map 3FDGal, 3FDGalA, and 3FDGtol. Slices of 3 mm thickness and an in-plane resolution of 2 mm×2 mm were acquired.

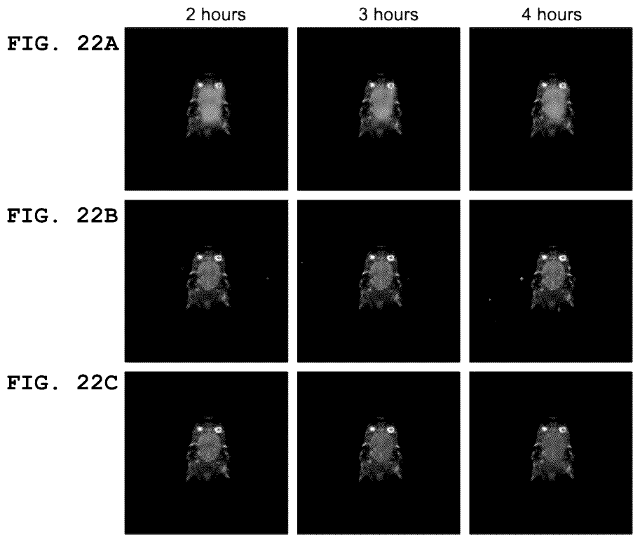

2D-FMI of the brain was also performed following the injection of 3FDG. 3FDG (0.6 g/kg) was intraperitoneally administered to healthy C57BL/6 male mice, and coronal mCSSI-based 2D-FMI was performed repeatedly, starting at t=60 min post-injection to map 3FDG (FIG. 22A, green colored overlay on 1H-MRI of the corresponding brain slice), 3FDGA (FIG. 22B, not shown in the brain), and 3FDS (FIG. 22C, red colored overlay on ¹H-MRI of the corresponding brain slice) simultaneously and noninvasively to visualize the real-time in vivo metabolism of 3FDG. The in vivo imaging data highlights the early, widespread uptake of the injected 3FDG, followed by metabolic reduction to 3FDGS, revealing their spatial distributions. Note that 3FDGA, the oxidized metabolite of 3FDG, was not found in the brain of healthy mice. MRI experiments were done on a 15.2 T MRI scanner with a 23 mm 1H/19F RF volume coil. A 20 mm single slice with in-plane resolution of 2 mm×2 mm was acquired at each 2D-FMI time point.

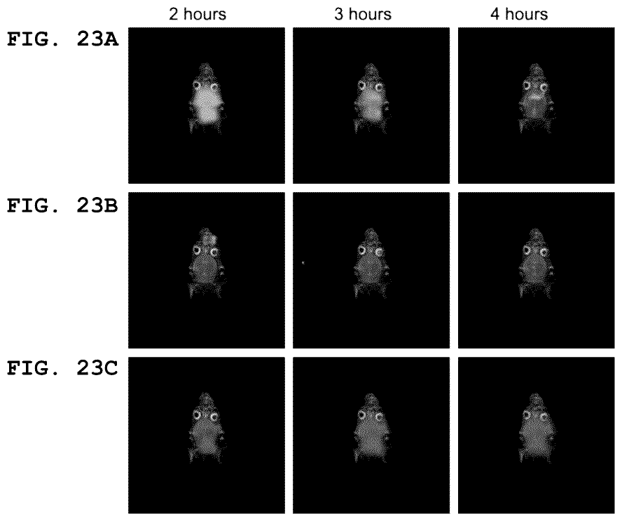

2D-FMI of the brain following the injection of 3FDGal was next conducted in healthy C57BL/6 male mice. 3FDGal (0.6 g/kg) was intraperitoneally administered to healthy C57BL/6 male mice, and coronal mCSSI-based 2D-FMI was performed repeatedly, starting at t=60 min post-injection to map 3FDGal (FIG. 23A, green colored overlay on ¹H-MRI of the corresponding brain slice), 3FDGalA (FIG. 23B, gold colored overlay on ¹H-MRI of the corresponding brain slice), and 3FDGtol (FIG. 23C, magenta colored overlay on ¹H-MRI of the corresponding brain slice) simultaneously and noninvasively to visualize the real-time in vivo metabolism of 3FDGal. The in vivo imaging data highlights the early, widespread uptake of the injected 3FDGal, followed by its disappearance and metabolic oxidation to 3FDGalA (first time point in the olfactory bulb ROI) and reduction to 3FDGtol, revealing their spatial distributions. MRI experiments were done on a 15.2 T MRI scanner with a 23 mm 1H/19F RF volume coil. A 20 mm single slice with in-plane resolution of 2 mm×2 mm was acquired at each 2D-FMI time point.

### Example 12

### Analysis of 3-F-Xylose Derivatives

Further to the analyses conducted for the ¹⁹F galactose derivative, 3FDXyl and its redox derivatives 3FDXA and 3FDXtol were also analyzed by NMR and their biodistribution was determined in vivo, using the same experimental procedures practiced above.

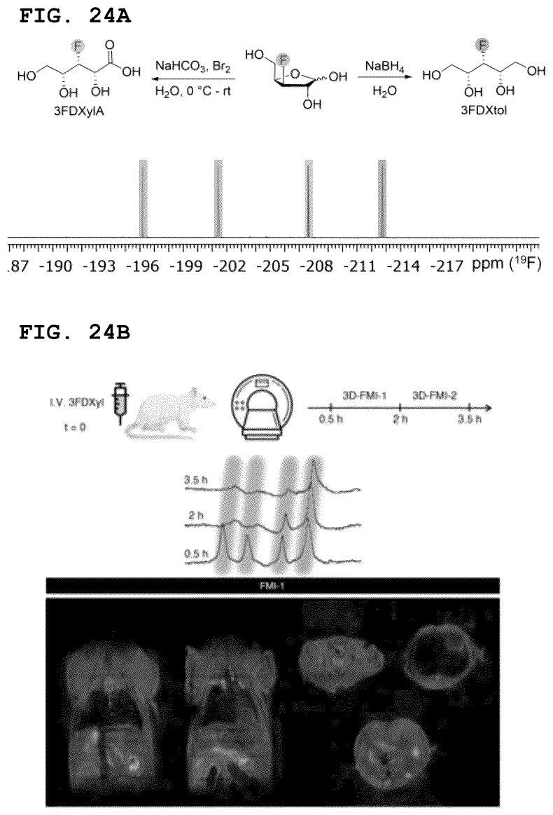

FIG. 24A shows 19F-NMR spectra of 3FDXyl and its redox derivatives (3FDXA, 3FDXtol) as well as their chemical structures. FIG. 24B shows a preliminary in vivo EMI after administration of 3FDXyl to healthy mice. The non-localized 19F-NMR acquired after probe injection shows the longitudinal transformation of 3FDXyl to its redox metabolites, and the FMI maps show the biodistribution of 3FDXtol (magenta) and 3FDXylA (gold).

### Example 13

### Developing fFMI Acquisition Protocol

Further optimization of the fFMI acquisition protocol was performed, improving both temporal resolution (reducing time acquisition from 80 to 20 minutes per step) and spatial resolution (decreasing voxel size from 2×2×3 mm to 1.5×1.5×3 mm, a ~44% reduction). These enhancements were achieved through bandwidth optimization: narrowing the receiver BW from 50 kHz to 10 kHz and reducing the excitation/refocusing BW from 2 kHz to 1 kHz.

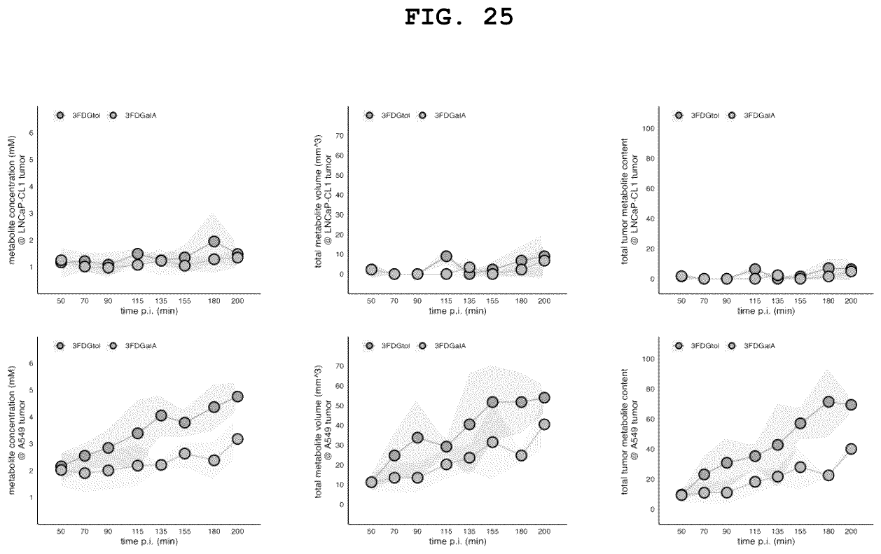

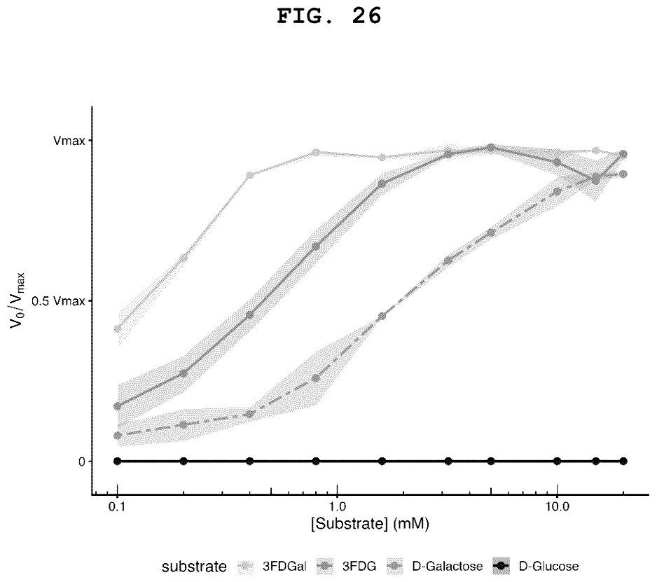

Data quantification is based on a phantom tube containing known concentrations of 3FDGalA and 3FDGtol and is shown in FIG. 25 for LNCaP-CL1 tumor in the upper row and A549 tumor in the lower row.

The data indicate that we visualize 3FDGtol concentration of 4-5 mM and 3FDGalA concentration of 2-3 mM within the A549 tumor. It may also be concluded that optimal tumor imaging occurs approximately three (3) hours post-injection.

### Example 14

### Determining the Affinity of AKR1B1 Enzyme for Different Sugars

AKR1B1 enzyme kinetics were measured in a 384-well plate(18 μL reaction volume per well) containing 20 μg enzyme and substrate (D-glucose, D-galactose, 3FDG, or 3FDGal) prepared in 100 mM phosphate buffer (pH7.2). Ten substrate concentrations were tested (0.1, 0.2, 0.4, 0.8, 1.6, 3.2, 5, 10, 15, and 20 mM), each in triplicate. Reactions were initiated at t=0 by adding 160p MNADPH (MedChemExpress, catalog no. HY-F0003) simultaneously to all wells. NADPH consumption was monitored spectrophotometrically at 340 nm every 76 s for 30 time points (~40 min total) using a Pherastar FS plate reader (BMG Labtech). Blank controls (AKR1B1+NADPH without substrate; n=3 wells) were averaged across all time points to define a normalization reference. Initial velocities(V₀) were computed from the second time point (1.27 min) as V₀=(blank reference-normalized readout)/1.27 (%/min). Triplicate wells per substrate concentration were averaged to obtain mean initial velocities±SD (n=10 concentrations/substrate). Michaelis-Menten parameters (Vmax,Km) were estimated separately for each substrate by nonlinear least squares in R(4.4.2), fitting V=Vmax·[S]/(Km+[S])using the nls function from stats(v4.4.2). Parameter extraction and data processing used tidyverse, broom, and ggh4x packages. Velocities were normalized to fitted substrate-specific Vmax(V₀/Vmax) for visualization. Mean±SD ribbons were plotted versus substrate concentration with linear and log₁₀ x-axes. All fits converged successfully.

Calculated Km: 3FDGal: 0.1 mM, 3FDG: 0.42 mM, D-Gal: 2.01 mM. The results indicate the high affinity of the AKR1B1 for 3FDGal.

1) A metabolic imaging method based on ¹⁹F-MRI, comprising the steps of:
administering one or more 3-fluoro-3-deoxy-sugar(s) to a subject;
applying ¹⁹F-magnetic resonance imaging (¹⁹F-MRI) to identify generation, localization and/or distribution of one or more fluorinated compounds in internal organs of the subject; and
assessing and/or diagnosing and/or monitoring a disease state associated with a pattern of abnormal activity of oxidizing and reducing metabolic enzymes.

2) A method according to claim 1, comprising identifying the generation, localization and/or distribution of one or more fluorinated compounds selected from:
the polyol metabolite of the 3-fluoro-3-deoxy-sugar,
downstream metabolites of said polyol;
the acid metabolite of the 3-fluoro-3-deoxy-sugar; and
downstream metabolites of said acid.

3) A method according to claim 2, comprising identifying at least one fluorinated compound selected from the group consisting of the polyol metabolite of the 3-fluoro-3-deoxy-sugar and downstream metabolite of the polyol; and at least one fluorinated compound selected from the group consisting of the acid metabolite of the 3-fluoro-3-deoxy-sugar or downstream metabolite of said acid; and
assessing and/or diagnosing and/or monitoring a disease state associated with a pattern of abnormal activity of oxidizing and reducing enzymes which are aldehyde dehydrogenase and aldose reductase, respectively.

4) A method according to claim 3, wherein the 3-fluoro-3-deoxy sugar is selected from 3-fluoro-3-deoxy-D-galactose, 3-fluoro-3-deoxy-D-glucose, and 3-Fluoro-3-deoxy-D-xylose.

5) A method according to claim 4, wherein the 3-fluoro-3-deoxy sugar is 3-fluoro-3-deoxy-D-galactose.

6) A method according to claim 3, wherein the ¹⁹F-magnetic resonance imaging is multiplexed/multispectral ¹⁹F-MRI, which comprises acquiring distinct ¹⁹F MR signals assigned to individual ¹⁹F-compounds consisting of the polyol and acid metabolites of said 3-fluoro-3-deoxy-sugar, and optionally the 3-fluoro-3-deoxy-sugar, in a single imaging session and simultaneously visualizing their formation in internal organs.

7) A method according to claim 6, wherein the acquisition of the distinct ¹⁹F MR signals comprises applying a pulse sequence that excites multiple resonances in a single imaging session and recording per-voxel multiple signal intensities, each corresponding to a spatial distribution of a specific frequency assigned to an individual fluorinated compound.

8) A method according to claim 7, comprising ¹⁹F-multi chemical shift selective imaging.

9) A method according to claim 1, further comprising creating with ¹H-MRI scan images of the internal organs of the examined subject, to identify the anatomical context of ¹⁹F-labeled cells, and producing a merged image based on the ¹H-MRI and ¹⁹F-MRI signals.

10) A method according to claim 1, the disease detected, assessed and/or monitored is a metabolic disease associated with abnormal expression of at least one of aldehyde dehydrogenase and aldose reductase.

11) A method according to claim 1, wherein the disease detected, assessed and/or monitored is cancer associated with overexpression of at least one of aldehyde dehydrogenase and aldose reductase.

12) A method according to claim 11, wherein the cancer is renal cell carcinoma, lung cancer, or brain tumor.

13) A method according to claim 11, wherein the 3-fluoro-3-deoxy sugar is 3-fluoro-3-deoxy-D-galactose, comprising recording and/or mapping signals at Δω=11.6±0.5 of Δω=6.8±0.5, relative to the signal of the beta anomer of 3-fluoro-3-deoxy-D-galactose, respectively, corresponding to the reduced and oxidized metabolites of 3-fluoro-3-deoxy-D-galactose.

14) A method of treating cancer in a patient, wherein the cancer is determined to be associated with elevated activity of at least one of aldehyde dehydrogenase and aldose reductase, comprising the steps of:
identifying generation, localization and/or distribution of one or more fluorinated compounds by the ¹⁹F-MRI metabolic imaging method of claim 11; wherein
when the ¹⁹F-MRI metabolic imaging indicates a localized disease, treating the patient by surgery and/or radiotherapy;
when the ¹⁹F-MRI metabolic imaging indicates a locally-advanced disease, treating the patient by surgery and/or radiotherapy; and
when the ¹⁹F-MRI metabolic imaging indicates an advanced, spread (metastatic) disease state, treating the patient by systemic administration of one or more active agents.

15) An injectable composition comprising an isotonic saline, sterile, optionally buffered solution of at least one of 3-fluoro-3-deoxy-D-galactose or 3-Fluoro-3-deoxy-D-xylose for use in ¹⁹F-MRI metabolic imaging for detecting a disease that exhibits abnormal pattern of oxidizing and/or reducing enzymes.

16) An injectable composition according to claim 15, comprising a buffer selected from phosphate, histidine and citrate.

17) An in vivo metabolic imaging method based on PET, comprising the steps of:
administering 3-¹⁸F-3-deoxy-sugar radiotracer selected from the group consisting of 3-¹⁸F-3-deoxy-D-galactose and 3-¹⁸F-3-deoxy-D-xylose to a subject;
scanning the subject with positron emission tomography (PET) to identify generation, localization and/or distribution of fluorinated compound(s) in internal organs and/or whole body of the subject; and
assessing and/or diagnosing and/or monitoring a disease state associated with a pattern of abnormal activity of oxidizing and/or reducing enzymes, which are aldehyde dehydrogenase and aldose reductase, respectively.

18) A method according to claim 17, wherein the 3-¹⁸F-3-deoxy-sugar is 3-¹⁸F-3-deoxy-D-galactose.

19) A method according to claim 18, wherein the disease state is a metabolic disease or cancer.

20) A method of treating cancer in a patient, wherein the cancer is determined to be associated with elevated activity of at least one of aldehyde dehydrogenase and aldose reductase, comprising the steps of:
identifying generation, localization and/or distribution of one or more fluorinated compounds by the PET of claim 17; wherein
when the PET indicates a localized disease, treating the patient by surgery and/or radiotherapy;
when the PET indicates a locally-advanced disease, treating the patient by surgery and/or radiotherapy; and
when the PET indicates an advanced, spread (metastatic) disease state, treating the patient by systemic administration of one or more active agents.

21) An injectable composition comprising an isotonic saline, sterile, optionally buffered solution of 3-¹⁸F-3-deoxy-D-galactose or 3-¹⁸F-3-deoxy-D-xylose.

22) An injectable composition according to claim 21, comprising a buffer selected from phosphate, histidine and citrate.
